(19) United States
(12) Patent Application Publication
(10) Pub. No.: US 2026/0318394 A1
(43) Pub. Date: Oct. 15, 2026

(54) Motion Converting Attachment For A Surgical Tool

(71) Applicant: Stryker Corporation, Portage, MI (US)

(72) Inventors: Gaurav Anerao, Mumbai (IN); Ankur Bhagat, Haldwani (IN); Ritesh Hooda, Rohtak (IN); Gursagar Singh, Amritsar (IN)

(73) Assignee: Stryker Corporation, Portage, MI (US)

(21) Appl. No.: 19/485,539

(22) PCT Filed: May 20, 2024
(86) PCT No.: PCT/US2024/030219

§ 371 Date: Nov. 18, 2025

Related U.S. Application Data
(60) Provisional application No. 63/467,505, filed on May 18, 2023.

(51) Int. Cl. F16H 37/16; A61B 17/16

(52) U.S. Cl. CPC F16H 37/16; A61B 17/1624

(57) ABSTRACT

An attachment for converting rotational motion of a rotary surgical tool to oscillating motion of a cutting accessory. The attachment includes a housing extending along a drive axis between a proximal end and a distal end and defining an interior cavity. The attachment further includes an input shaft and an output shaft each supported for rotation about the drive axis and at least partially disposed in the interior cavity. The attachment further includes a crank shaft supported for rotation about a crank axis and rotationally coupled to the input shaft. The crank axis is perpendicular to the drive axis. The attachment further includes a rocker shaft supported for rotation about a rocker axis and rotationally coupled to the output shaft. The attachment further includes an intermediate link coupled between the crank shaft and the rocker shaft.

### CROSS-REFERENCE TO RELATED APPLICATIONS

The subject patent application claims priority to and all the benefits of U.S. Provisional Patent Application No. 63/467,505, filed on May 18, 2023, the entire contents of which are incorporated by reference herein.

### BACKGROUND

Conventional medical procedures routinely involve the use of surgical tools to assist medical professionals in approaching, viewing, manipulating, or otherwise effecting treatment at localized surgical sites. Some of these medical procedures may involve surgical techniques such as drilling, shaping, or decortication of bone using a rotary instrument, where a cutting accessory, such as a high-speed bur, rotates at speeds in excess of 75 k rpm to remove tissue. During use, contact between non-cutting portions of the bur (e.g., a shank) and soft tissue can result in tissue wrap, in which friction between the rotating bur shank and the soft tissue causes the soft tissue to be pulled around the shank. Surgeons must be mindful to avoid unexpected tissue wrap.

### SUMMARY

In a first aspect, an attachment for a rotary surgical tool comprises a housing extending along a drive axis between a proximal end and a distal end and defining an interior cavity. The attachment further comprises an input shaft and an output shaft each supported for rotation about the drive axis and at least partially disposed in the interior cavity. The attachment further comprises a crank shaft supported for rotation about a crank axis and rotationally coupled to the input shaft. The crank axis is perpendicular to the drive axis. The attachment further comprises a rocker shaft supported for rotation about a rocker axis and rotationally coupled to the output shaft. The attachment further comprises an intermediate link coupled between the crank shaft and the rocker shaft.

In a second aspect, an attachment for a rotary surgical tool comprises a housing extending between a proximal end and a distal end and defining an interior cavity. The attachment further comprises an input shaft supported for rotation in the housing and having an input bevel gear arranged at a distal end of the input shaft. The attachment further comprises an output shaft supported for rotation in the housing and having an output bevel gear arranged at a proximal end of the output shaft. The attachment further comprises an oscillatory motion linkage operably coupled between the input shaft and the output shaft. The oscillatory motion linkage comprises a crank shaft, a rocker shaft, and an intermediate link. The crank shaft is supported for rotation about a crank axis. The rocker shaft is supported for rotation about a rocker axis. The intermediate link is coupled between the crank shaft and the rocker shaft and configured to convert rotational movement of the crank shaft to oscillatory movement of the rocker shaft. The oscillatory motion linkage further comprises a crank bevel gear coupled to the crank shaft and engaged with the input bevel gear. The oscillatory motion linkage further comprises a rocker bevel gear coupled to the rocker shaft and engaged with the output bevel gear.

In a third aspect, an attachment for a rotary surgical tool comprises a housing assembly extending along a drive axis between a proximal end and a distal end. The housing assembly comprises a first housing body and a second housing body coupled to the first housing body at a mid-plane. The mid-plane is parallel to the drive axis. An interior cavity is defined by the first housing body and the second housing body. The attachment further comprises an input shaft and an output shaft each supported for rotation about the drive axis and at least partially disposed in the interior cavity. The attachment further comprises a crank shaft, a rocker shaft, and an intermediate link. The crank shaft is rotationally coupled to the input shaft. The rocker shaft is rotationally coupled to the output shaft. The intermediate link is coupled between the crank shaft and the rocker shaft and arranged for coordinated movement along a plane parallel to the mid-plane.

In a fourth aspect, an attachment for a rotary surgical tool comprises a housing extending between a proximal end and a distal end and defining an interior cavity. The attachment further comprises an input shaft, and output shaft, and an oscillatory motion linkage. The input shaft is supported for rotation in the housing. The output shaft is supported in the housing coaxial to the input shaft. The oscillatory motion linkage is operably coupled between the input shaft and the output shaft and disposed in the interior cavity. The oscillatory motion linkage is configured to convert rotational motion of the input shaft to oscillating motion of the output shaft.

In a fifth aspect, a surgical bur system comprises a rotary surgical handpiece, an oscillatory motion attachment, and a nose tube assembly. The rotary surgical handpiece comprises a motor, an attachment interface, and a motor shaft arranged in the attachment interface for supplying rotational motion from the motor. The oscillatory motion attachment is removably couplable with the rotary surgical handpiece and comprises a housing extending between a proximal end and a distal end and configured to engage the attachment interface. The oscillatory motion attachment further comprises an input shaft supported for rotation in the housing and arranged to engage the motor shaft when the housing is engaged with the attachment interface. The oscillatory motion attachment further comprises an output shaft supported in the housing coaxial to the input shaft. The oscillatory motion attachment further comprises an oscillatory motion linkage operably coupled between the input shaft and the output shaft and disposed in the interior cavity. The oscillatory motion linkage is configured to convert rotational motion of the input shaft to oscillating motion of the output shaft. The nose tube assembly removably coupled to the oscillatory motion attachment opposite the rotary surgical handpiece. The nose tube assembly comprises a drive shaft arranged to engage the output shaft.

In a sixth aspect, an attachment for a rotary surgical tool. comprises a housing extending between a proximal end and a distal end. The housing defines an interior cavity. The attachment also includes an input shaft supported for rotation along a first axis in the housing. The attachment also includes an output shaft supported in the housing for rotation along a second axis in the housing intersecting the first axis. The attachment also includes an oscillatory motion linkage operably coupled between the input shaft and the output shaft and disposed in the interior cavity, where the oscillatory motion linkage is configured to convert rotational motion of the input shaft to oscillating motion of the output shaft.

In a seventh aspect, a rotary surgical tool including an oscillatory conversion mechanism, an input shaft, and an output shaft comprises a cam shaft supported for rotation about a cam axis and configured to be coupled to the input shaft. The cam shaft may include, a proximal end configured to be coupled to the input shaft, a clockwise cam lobe, and a counter-clockwise cam lobe axially spaced from the clockwise cam lobe, where the cam lobes are configured to revolve in the same direction about the cam axis as the cam shaft is rotated about the cam axis. The tool also includes a rocker shaft supported for rotation about a rocker axis and configured to be coupled to the output shaft, the rocker axis being parallel to the cam shaft. The rocker shaft may include: a first cam follower to abut the clockwise cam lobe to rotate the rocker shaft in a first direction about the rocker axis, and a second cam follower axially spaced from the first cam follower to abut the counter-clockwise cam lobe to rotate the rocker shaft in a second direction about the rocker axis opposite the first direction. The cam followers alternatingly abut the cam lobes to oscillate the rocker shaft.

Any of the above aspects can be combined in full or in part. Any features of the above aspects can be combined in full or in part. Any of the above implementations for any aspect can be combined with any other aspect. Any of the above implementations can be combined with any other implementation whether for the same aspect or a different aspect.

### BRIEF DESCRIPTION OF THE DRAWINGS

Advantages of the present disclosure will be readily appreciated as the same becomes better understood by reference to the following detailed description when considered in connection with the accompanying drawings.

FIG. 1 is a perspective environmental view of an exemplary surgical system in use by a surgeon, the surgical system is shown having a surgical tool, an attachment, a nose tube assembly, and a cutting accessory.

FIG. 2 is a perspective view of the surgical tool, the attachment, the nose tube assembly, and the cutting accessory of FIG. 1.

FIG. 3 is an exploded view of the surgical tool, the attachment, the nose tube assembly, and the cutting accessory of FIG. 1.

FIG. 4 is a cross-sectional view of the attachment and the nose tube assembly taken along line 4-4 of FIG. 5.

FIG. 5 is another cross-sectional view of the attachment and the nose tube assembly taken along line 5-5 of FIG. 3.

FIG. 6 is an exploded view of the attachment and the nose tube assembly of FIGS. 4 and 5 showing an oscillatory motion linkage.

FIG. 7 is an exploded view of the attachment and the oscillatory motion linkage of FIG. 6 showing a crank shaft, a rocker shaft, and an intermediate link.

FIGS. 8 and 9 are exploded views of the oscillatory motion linkage of FIG. 7 showing the crank shaft, the rocker shaft, and the intermediate link.

FIG. 10 is a simplified side view of the oscillatory motion linkage of FIGS. 7-9 showing the crank shaft, the rocker shaft, and the intermediate link.

FIG. 11 is a cross-sectional view of the simplified oscillatory motion linkage of FIG. 10.

FIGS. 12 and 13 are exploded views of the oscillatory motion linkage showing the rocker shaft, the intermediate link, and a second implementation of the crank shaft.

FIG. 14 is a perspective view of a third implementation of the oscillatory motion linkage.

FIG. 15 is a sectional view of the third implementation of the oscillatory motion linkage.

FIG. 16 is a perspective view of a fourth implementation of the oscillatory motion linkage.

FIG. 17 is a sectional view of the fourth implementation of the oscillatory motion linkage.

FIG. 18 is a sectional view of a fifth implementation of the oscillatory motion linkage.

FIG. 19 is a perspective view of a second attachment including an oscillatory conversion mechanism.

FIG. 20 is a sectional view of the second attachment.

FIG. 21 is a perspective view of the oscillatory conversion mechanism of the second attachment.

FIG. 22 is an exploded view of a rocker shaft of the oscillatory conversion mechanism.

FIG. 23 is a perspective view of a cam shaft of the oscillatory conversion mechanism.

FIG. 24 is a side elevation view of the oscillatory conversion mechanism.

FIG. 25A is a sectional view of the oscillatory conversion mechanism in a first orientation taken along lines 25A-25A of FIG. 24.

FIG. 25B is a sectional view of the oscillatory conversion mechanism in the first orientation taken along lines 25B-25B of FIG. 24.

FIG. 25C is a sectional view of the oscillatory conversion mechanism in the first orientation taken along lines 25C-25C of FIG. 24.

FIG. 26A is a sectional view of the oscillatory conversion mechanism in a second orientation taken along lines 25A-25A of FIG. 24.

FIG. 26B is a sectional view of the oscillatory conversion mechanism in the second orientation taken along lines 25B-25B of FIG. 24.

FIG. 26C is a sectional view of the oscillatory conversion mechanism in the second orientation taken along lines 25C-25C of FIG. 24.

### DETAILED DESCRIPTION

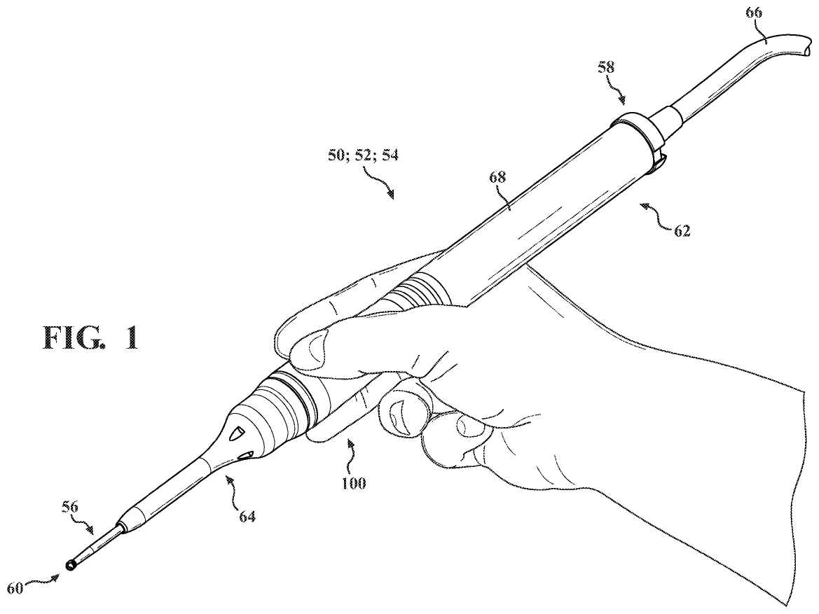

Referring now to the drawings, wherein like numerals indicate like parts throughout the several views, a surgical system 50 is shown in FIG. 1. The surgical system 50 generally comprises a surgical instrument 52, and in some implementations may further comprise additional accessories usable with the surgical instrument 52. For example, the surgical system may further comprise an irrigation system (not shown), a navigation system (not shown), and the like. In the representative embodiment illustrated herein, the surgical instrument 52 is realized as a surgical tool 54, and more specifically, a rotary surgical tool. The surgical system 50 may further comprise a console (not shown) may be employed to control the surgical instrument 52 via a footswitch. However, the surgical instrument 52 could be configured and controlled in a number of different ways. By way of non-limiting example, the surgical instrument 52 could be controlled independently, such as by a discrete console or input devices.

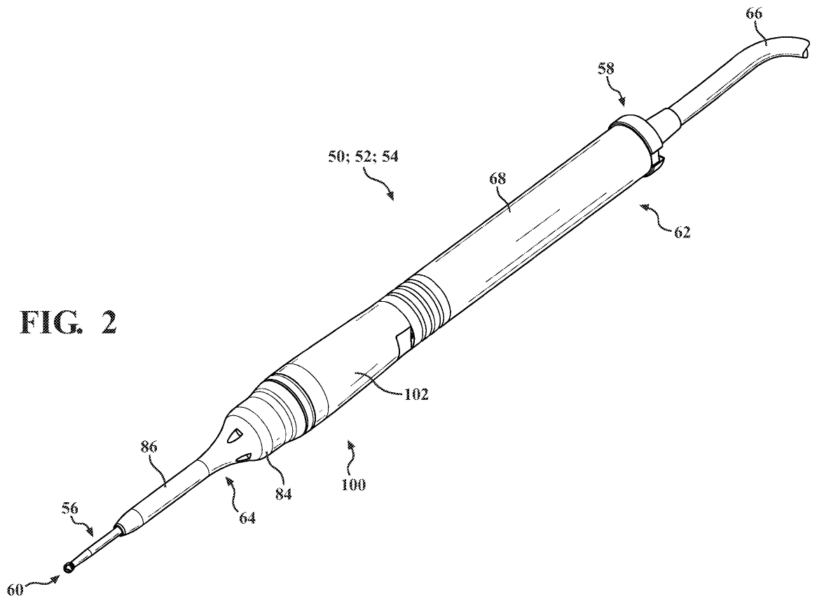

Referring still to FIGS. 1 and 2, the surgical instrument 52 is configured as the rotary surgical tool 54, which drives a cutting accessory, generally indicated at 56. Here, the cutting accessory 56 is adapted to assist a medical professional in performing a surgical procedure by effecting the removal of tissue, bone, and the like. To this end, the cutting accessory 56 is depicted in FIGS. 1-5 as a bur. Alternatively, the cutting accessory 56 could be of a number of different types or configurations, such as drills, shavers, rasps, ultrasonic cutting tools, etc.

In FIG. 2, a perspective view of a first configuration of the surgical instrument 52 is shown. The surgical instrument 52 has a proximal end 58 and a distal end 60 that are spaced along an instrument axis A1. As used herein, “distal” generally refers to portions that are closer to the patient during use (e.g., a material removal tool), and “proximal” generally refers to portions that are further from the patient during use (e.g., a power cord). Typically, a surgical instrument transfers mechanical energy along an instrument axis Al from a source (e.g., a motor or an ultrasonic transducer) arranged near a proximal end to an attachment coupled to a distal end of the surgical instrument.

The surgical instrument 52 may comprise an attachment 100, a surgical handpiece 62 (or handpiece), a nose tube assembly 64, and the cutting accessory 56. The attachment 100 is configured to engage the surgical handpiece 62 and to receive the cutting accessory 56. The attachment 100 transfers rotary mechanical energy from the handpiece 62 to the cutting accessory 56. The handpiece 62 is powered and may receive electrical control signals from, for example, a control system coupled to the handpiece 62 by way of a flexible supply cable 66.

The handpiece 62 may include a housing 68, a motor 70 disposed in the housing 68, a flexible supply cable 66 protruding from the housing 68 in a proximal region, and an attachment interface 72 near a distal end of the housing 68. Exemplary surgical instruments can be found in U.S. Pat. Nos. 8,597,316 and 10,537,339, which are incorporated by reference in their entirety herein.

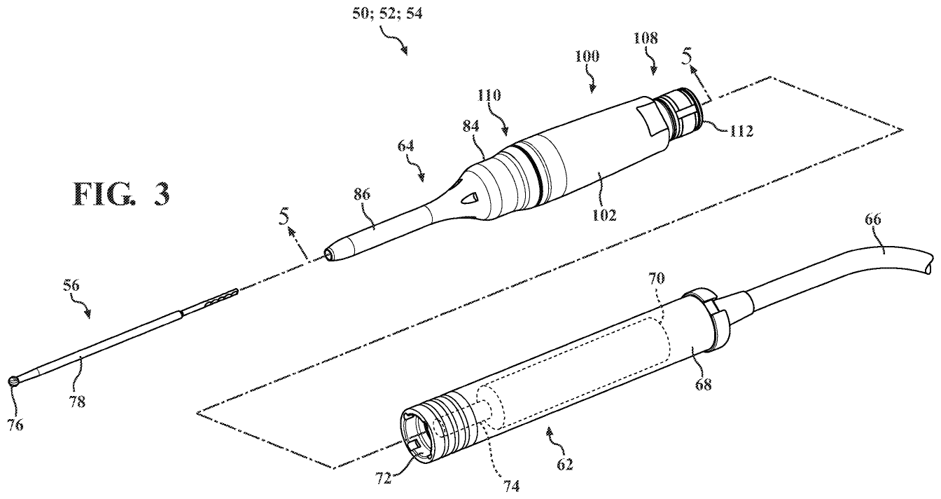

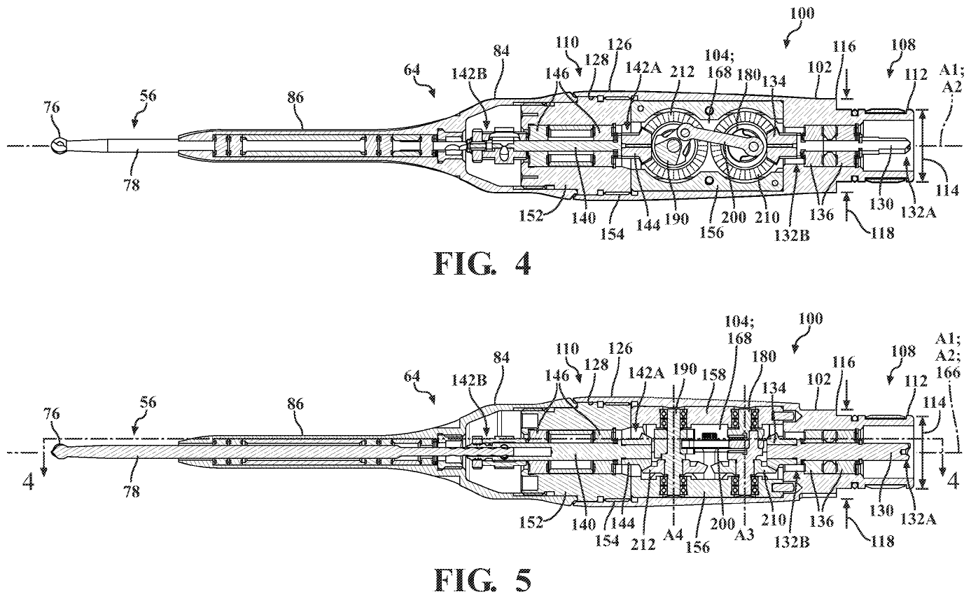

The motor 70 generates rotational motion at a motor shaft 74, which is arranged in the attachment interface 72 for supplying the mechanical energy from the motor 70 to the cutting accessory 56 or attachment. As mentioned above, the surgical instrument 52 may comprise the nose tube assembly 64, which may support the cutting accessory at a distal end. As shown in FIG. 3, the cutting accessory 56 may comprise a head 76 and a shank 78 extending from the head 76. The shank 78 is adapted to be rotatably supported by the nose tube assembly 64 and is secured axially to the attachment 100 via a tool chuck 80 (FIG. 4). In connection with the exemplary cutting accessory 56 illustrated herein, the head 76 is realized as a bur, but could be of any suitable type or configuration (e.g., a drill), as noted above.

In the exemplary surgical instrument 52 illustrated in FIGS. 1-3, the motor 70 is powered via a wired electrical connection with the console (not shown) and is controlled via a footswitch, which is similarly disposed in electrical communication with the console. However, the surgical instrument 52 could be configured with or without a wired motor 70 controlled by a console. By way of non-limiting example, the surgical instrument 52 could be powered pneumatically or could be driven by a motor disposed within the console. Similarly, while the footswitch is employed to effect control of the motor 70 via the console, other types of user inputs are contemplated. For example, hand switches could be operatively attached to the housing 68 of the surgical instrument 52 to control rotation of the motor 70, or the console could control rotation of the motor 70 without the footswitch.

When removing tissue with the cutting accessory 56, loose material may adhere to the shank 78 and cause tissue wrap. Specifically, when the cutting accessory 56 and shank 78 are spinning at high rotational speeds, flutes on the head 76 cause soft tissue to take turns around the shank 78, surface friction with the shank 78 causes the soft tissue to adhere to the shank 78 and cause tissue wrap, which may damage soft tissue and clog the nose tube assembly 64. Furthermore, accumulated tissue may reduce the surgeon's line of sight to the surgical site. One method for reducing tissue wrap is to alter the motion of the cutting accessory 56. Some types of cutting accessories and the associated motion are more prone to tissue wrap than other cutting accessories that have a different motion. One type of cutting accessory 56 that tends to cause tissue wrap is a rotary tool with a small diameter shank. Because certain procedures require a tool with certain physical characteristics (e.g., shank diameter), the motion of the cutting accessory is altered to reduce tissue wrap. For example, another type of cutting accessory 56 is an oscillatory cutting accessory. Here, the oscillatory cutting accessory oscillates or pivots about a tool axis. In one exemplary implementation of the oscillatory cutting accessory, the head 76 and the shank 78 may oscillate between two positions that are separated by 180 degrees. Said differently, the cutting accessory 56 has an oscillatory motion of ±90 degrees. This reduces potential for trauma due to tissue wrap during use. In some implementations the cutting accessory 56 may be optimized for different oscillation amounts based on, for example, the geometry of the head 76. As such, the cutting accessory 56 may have an oscillatory motion of ±15 degrees, ±30 degrees, ±45 degrees, ±60 degrees, ±135 degrees, ±180 degrees, and ranges other than those specifically enumerated.

As mentioned above, certain procedures are advantageously performed with an instrument having a certain size, configuration, or form factor. For example, minimally invasive surgeries are performed with an instrument having a small diameter and a long length. Such instruments are typically hand-held with a pencil-like grip by a surgeon, and as such a consistent configuration of the instrument with a well-balanced weight is further advantageous. In order to provide a surgical instrument 52 capable of implementing the advantages described above, the surgical system 50 further comprises an attachment 100 for converting rotational motion into oscillatory motion. The attachment 100 is removably coupled to the handpiece 62 for operation therewith.

Some implementations of the surgical instrument 52 may include a nose tube assembly 64 that is removably coupled to the attachment 100. As will be described in greater detail below, the nose tube assembly 64 may comprise a nose housing 84, a nose tube 86, and a drive shaft. The drive shaft is rotatable about a nose tube axis, which is illustrated herein as coaxial with the instrument axis A1, for transferring power from the handpiece 62 to the cutting accessory 56. The tool chuck 80 is supported for rotation in the nose housing 84 and configured to receive the cutting accessory 56 arranged at a working end of the surgical instrument 52. Here, the working end refers generally to a distal-most portion of the surgical instrument 52 intended to perform work (e.g., material removal) on the patient. The tool chuck 80 is operably coupled to the drive shaft to receive power from the handpiece 62. The nose housing 84 may include a threaded neck 90 at a proximal end configured for removable engagement with the attachment 100. A distal end of the nose housing 84 is coupled to the nose tube 86, which may have a reduced diameter to aid access to a surgical site.

In some implementations, the nose tube assembly 64 may be angled relative to the instrument axis A1. Said differently, the nose tube axis is angled relative to the instrument axis A1. Such a nose tube assembly may facilitate better control and/or visibility by the surgeon during use. For example, the surgeon may prefer that the working end be angled when performing procedures with one type of cutting tool while preferring that the working end be straight when performing procedures with another type of cutting tool. The nose tube assembly 64 may be straight, or angled at an angle such as 15 degrees, 45 degrees, etc. Additionally, the nose tube 86 may be of various lengths such as 30 mm, 50 mm, etc.

Illustrated in FIGS. 3-5, power from the handpiece 62 is transferred to the attachment 100 to drive the cutting accessory 56. The attachment 100 may comprise a housing 102 extending along a drive axis A2 between a proximal end 108 to a distal end 110 and defining an interior cavity 104. The proximal end 108 of the attachment 100 is configured for engaging the handpiece 62. The housing 102 may comprise a bushing portion 112 arranged at the proximal end 108 of the housing 102, which is engageable with a distal end of the handpiece 62. The bushing portion 112 is received by the handpiece 62 and is generally aligned with the instrument axis A1. The bushing portion 112 has a first diameter 114 that is sized to engage with the attachment interface 72 of the handpiece 62. The housing 102 may further comprise a mount flange 116 arranged distally of the bushing portion 112 and having a second diameter 118. The second diameter 118 of the mount flange 116 is greater than the first diameter 114 of the bushing portion 112. The second diameter 118 is sized so as to limit the insertion of the bushing portion 112 into the handpiece 62. The mount flange 116 abuts the distal end of the handpiece 62 when the attachment 100 is fully coupled to the handpiece 62.

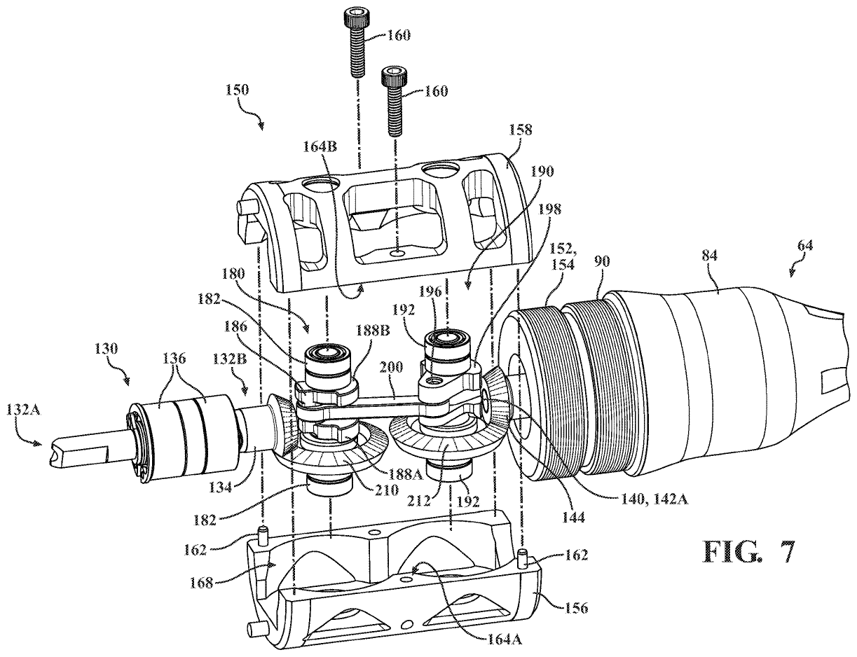

At the distal end 110 of the housing 102, opposite to the bushing portion 112, the housing further comprises an output portion 126 for engaging the nose tube assembly 64. The nose tube assembly 64 is removably coupled to attachment 100 at the distal end 110 of the housing 102. As shown best in FIG. 6, the output portion 126 is implemented as a threaded socket 128, which is configured to receive the corresponding threaded neck 90 of the nose tube assembly 64.

The attachment 100 further comprises an input shaft 130, an output shaft 140, and an oscillatory motion linkage 150. The input shaft 130 and the output shaft 140 are each at least partially disposed in the interior cavity 104 at opposing ends of the housing 102 and are supported for rotation. The input shaft 130 extends between a proximal end 132A and a distal end 132B and is supported for rotation in the housing 102. The output shaft 140 extends between a proximal end 142A and a distal end 142B and is supported for rotation in the housing 102. As will be discussed in further detail below, the oscillatory motion linkage 150 is operably coupled between the input shaft 130 and the output shaft 140. The distal end 132B of the input shaft 130 is engaged with a proximal end of the oscillatory motion linkage 150, and a distal end of the oscillatory motion linkage 150 is engaged with a proximal end 142A of the output shaft 140.

A corresponding gear may be coupled to each of the input shaft 130 and the output shaft 140 for operable engagement with the oscillatory motion linkage 150. An input gear 134 and an output gear 144 are coupled to the corresponding shaft for rotation therewith. Specifically, the input gear 134 may be coupled to, or adjacent to, the distal end 132B of the input shaft 130. Similarly, the output gear 144 may be coupled to, or adjacent to, the proximal end 142A of the output shaft 140. The corresponding gears and shafts may be coupled using an interference fit or secured for rotation with a set screw (not shown) threaded into the gear. Alternatively, a splined interface or other non-circular interface (e.g., a key) may be utilized to prevent relative rotation.

In the implementation illustrated herein the input gear 134 and the output gear 144 are illustrated as bevel gears, however other types of gears such as spiral bevel gears, hypoid gears, and crown gears may be utilized. Furthermore, the bevel gears are illustrated having a pitch angle of 45 degrees such that power is transferred at a right angle to the corresponding input shaft 130 or output shaft 140. It is contemplated that other pitch angles may be utilized to achieve a power transfer angle other than 90 degrees. Said differently, the input shaft 130 and the output shaft 140 are illustrated as being parallel with one another, the oscillatory motion linkage 150 may be configured to engage with the input shaft 130 and the output shaft 140 which are not parallel to one another. More specifically, the input shaft 130 and the output shaft 140 are shown as being coaxial with one another and supported for rotation about the drive axis A2 at least partially within the interior cavity 104 of the housing 102. One or more input bearings 136 may support the input shaft 130 in the housing 102. Similarly, one or more output bearings 146 may support the output shaft 140 in the housing 102.

Turning now to the partially exploded view of FIG. 6, the attachment 100 is shown with the nose tube assembly 64 spaced from the housing 102 and with the oscillatory motion linkage 150 removed from the housing 102. Here, the attachment 100 is shown with an output shaft subassembly 148 arranged between the nose tube assembly 64 and the oscillatory motion linkage 150. The exemplary output shaft subassembly 148 illustrated here comprises the output shaft 140, the tool chuck 80, the output bearings 146, and an output shaft housing 152. The output shaft housing 152 houses the output bearings 146 that support the output shaft 140 for rotation about the drive axis A2. The output shaft housing 152 has a threaded portion 154 configured with engagement with the threaded socket 128 at the output portion 126 of the housing 102 to retain the output shaft 140 and output gear 144 in engagement with the oscillatory motion linkage 150. Here, the tool chuck 80 is shown coupled to the output shaft 140 to transfer motion to a cutting accessory that is disposed in the nose tube 86 and received in the tool chuck 80.

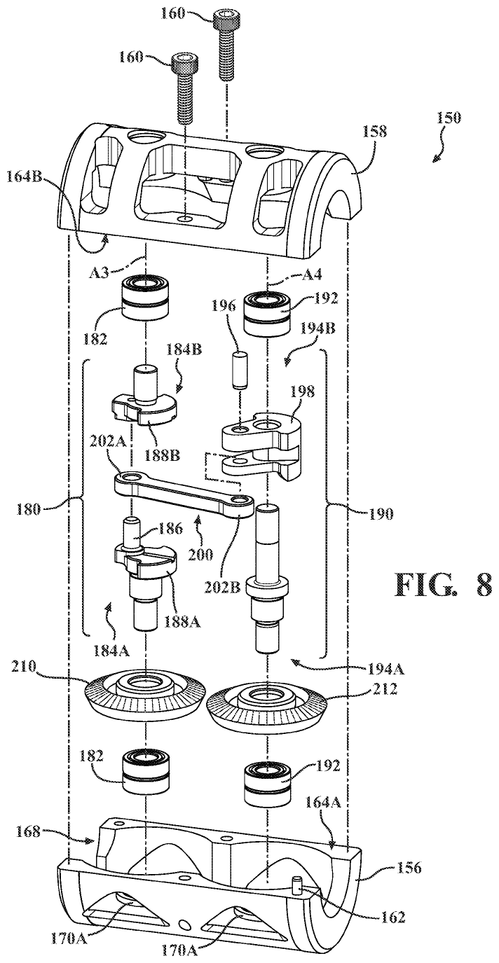

FIG. 7 shows another exploded view with the housing 102 removed to show engagement between the oscillatory motion linkage 150 and each of the input shaft 130 and the output shaft 140. The oscillatory motion linkage 150 may comprise a first housing body 156 and a second housing body 158, which cooperate to support operation of a crank shaft 180 and a rocker shaft 190, as will be discussed in further detail below. The first housing body 156 may be coupled to the second housing body 158 using fasteners 160, which clamp the first housing body 156 to the second housing body 158, and dowels 162, which facilitate precise alignment between the first housing body 156 and the second housing body 158. While not identical, the first housing body 156 and the second housing body 158 are generally similar with each having a mating surface 164, engagement of which defines a mid-plane 166. The first housing body 156 has a first mating surface 164A and the second housing body 158 has a second mating surface 164B. When the first mating surface 164A is engaged with the second mating surface 164B to couple the first housing body 156 to the second housing body 158, the mid-plane 166 is defined therebetween. Further, when the first housing body 156 is coupled to the second housing body 158, an interior cavity 168 is defined therein. In the exemplary embodiment illustrated herein, the mid-plane 166 is parallel to the drive axis A2; however, other configurations are contemplated. For example, implementations in which the input shaft 130 and the output shaft 140 are non-parallel may define the drive axis A2 differently such that the mid-plane 166 and the drive axis are non-parallel.

Each of the first housing body 156 and the second housing body 158 comprises a pair of bearing seats 170, a first pair of bearing seats 170A is arranged on the first housing body 156 and a second pair of bearing seats 170B is arranged on the second housing body 158. As will be discussed below, the bearing seats 170 support crank bearings 182 and rocker bearings 192, which in turn support the crank shaft 180 and the rocker shaft 190 for movement about their respective axes. The first pair of bearing seats 170A is arranged across the mid-plane 166 from the second pair of bearing seats 170B and defines an axis (the crank axis A3 and/or the rocker axis A4) that is perpendicular to the mid-plane 166.

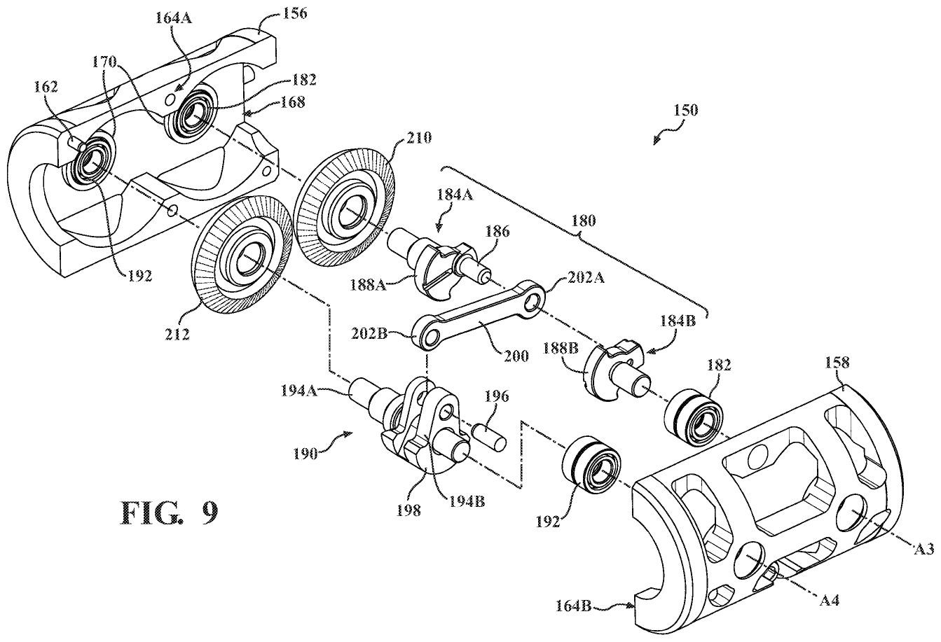

Turning now to FIGS. 7-9, further elements of the oscillatory motion linkage 150 are shown. The oscillatory motion linkage 150 comprises a crank shaft 180, a rocker shaft 190, and an intermediate link 200. The crank shaft 180 is supported for rotation about a crank axis A3 and rotationally coupled to the input shaft 130. Similarly, the rocker shaft 190 is supported for rotation about a rocker axis A4 and rotationally coupled to the output shaft 140. In the exemplary implementation illustrated herein, the crank axis A3 is parallel to the rocker axis A4. As mentioned above, the crank shaft 180 is supported in the interior cavity 168 by the first housing body 156 and the second housing body 158 on crank bearings 182, which are arranged in one of the bearing seats 170 in each of the first housing body 156 and the second housing body 158. Similarly, the rocker shaft 190 is supported in the interior cavity 168 by the first housing body 156 and the second housing body 158 on rocker bearings 192, which are arranged in the other one of the bearing seats 170 in each of the first housing body 156 and the second housing body 158.

The crank shaft 180 may include a first crank portion 184A and a second crank portion 184B, which are assembled to form the crank shaft 180. The crank shaft 180 further includes an offset joint 186 axially spaced from the crank axis A3 that is coupled to the intermediate link 200. The first crank portion 184A may be assembled to the second crank portion 184B to form the offset joint 186 of the crank shaft 180. As will be discussed in further detail below, during operation the offset joint 186 of the crank shaft 180 moves in a circular path around the crank axis A3. To this end, the crank shaft 180 may further include a counterweight 188 radially opposed from the offset joint 186. More specifically, the counterweight 188 includes a first counterweight 188A coupled to the first crank portion 184A and a second counterweight 188B coupled to the second crank portion 184B. During operation, the counterweight 188 dynamically balances the rotating mass of the offset joint 186 and the intermediate link 200 to reduce noise and vibration. Here, the first and second counterweights 188A, 188B are integrally formed with the respective crank portion 184A, 184B. In other implementations (not shown) the counterweight may be coupled to the crank shaft using, for example, fasteners or an interference fit.

The rocker shaft 190 may include a first rocker portion 194A and a second rocker portion 194B, which are assembled to form the rocker shaft 190. The rocker shaft 190 further includes an offset joint 196 axially spaced from the rocker axis A4 that is coupled to the intermediate link 200. The first rocker portion 194A may be assembled to the second rocker portion 194B to form the rocker shaft 190. Unlike the crank shaft 180, the offset joint 196 of the rocker shaft 190 is a shaft that is assembled to the second rocker portion 194B using, for example, an interference fit. As will be discussed in further detail below, during operation the offset joint 196 of the rocker shaft 190 moves in an arcuate path aligned with the rocker axis A4. To this end, the rocker shaft 190 may further include a counterweight 198 radially opposed from the offset joint 196. More specifically, the counterweight 198 is integrally formed with the second rocker portion 194B. During operation, the counterweight 198 dynamically balances the oscillating mass of the offset joint 196 and the intermediate link 200 to reduce noise and vibration. In other implementations (not shown) the counterweight may be coupled to the rocker shaft using, for example, fasteners or an interference fit.

As mentioned above, the input shaft 130 is rotationally coupled to the crank shaft 180 and the output shaft 140 is rotationally coupled to the rocker shaft 190. The input gear 134 is coupled to the input shaft 130 and the output gear 144 is coupled to the output shaft 140. To this end, a crank gear 210 is coupled to the crank shaft 180 and engaged with the input gear 134 to transfer rotational movement therebetween. Similarly, a rocker gear 212 is coupled to the rocker shaft 190 and engaged with the output gear 144 to transfer rotational movement therebetween. As with the input gear 134 and the output gear 144 described above, the crank gear 210 and the rocker gear 212 may be bevel gears. Indeed, in the exemplary implementation illustrated herein the crank gear 210 and the rocker gear 212 are bevel gears configured to transfer mechanical movement (rotation or oscillation, respectively) with the corresponding input gear 134 and output gear 144.

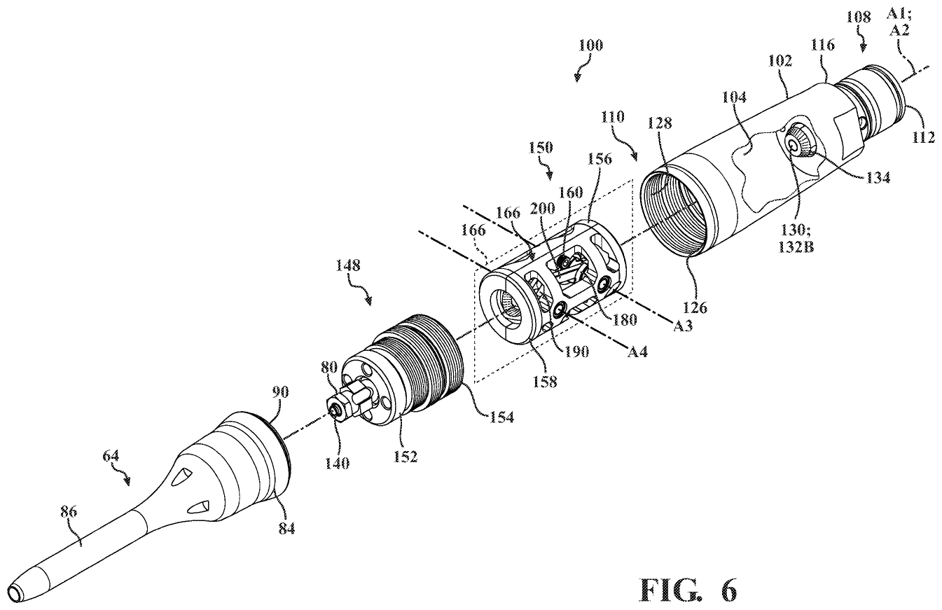

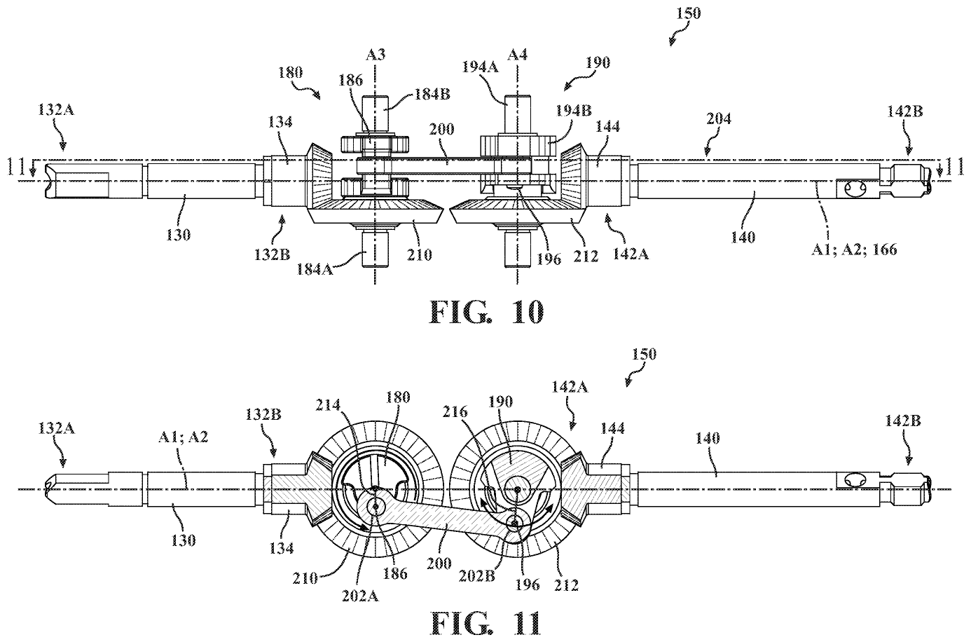

As shown in FIGS. 5 and 10, the crank gear 210 is coupled to the first crank portion 184A of the crank shaft 180 and is arranged on a first side of the mid-plane 166. Likewise, the rocker gear 212 is coupled to the first rocker portion 194A of the rocker shaft 190 and is arranged on the first side of the mid-plane 166. By reducing the height and the width of the oscillatory motion linkage with the input gear 134, crank gear 210, rocker gear 212, and output gear 144, the overall diameter of the attachment 100 can be reduced. Similarly, the ratios of speed of the motor 70 and the cutting accessory 56 can be optimized. A ratio between the input gear 134 and the crank gear 210 is approximately 2:1, which reduces the speed of the crank shaft 180 relative to the input shaft 130. Similarly, a ratio between the rocker gear 212 and the output gear 144 is approximately 2:1, which doubles the oscillation excursion (e.g., from ±45 degrees to ±90 degrees). Other implementations of the oscillatory motion linkage (not shown) may utilize ratios such as 1:1, 3:1, 4:1, etc. These ratios may have the effect of increasing or decreasing the relative rotational speed of the crank shaft 180 and the rocker shaft 190 as well as changing the associated stresses in the intermediate link 200.

With renewed reference to FIGS. 7-9, the intermediate link 200 is shown. The intermediate link 200 is coupled between the crank shaft 180 and the rocker shaft 190 and configured to convert rotational movement of the crank shaft 180 to oscillatory movement of the rocker shaft 190. The intermediate link 200 has two link eyes 202. A first link eye 202A is coupled to the offset joint 186 of the crank shaft 180 and a second link eye 202B is coupled to the offset joint 196 of the rocker shaft 190. During operation, movement of the intermediate link 200 follows that of a rocker-crank four-bar linkage. Rotation of the crank shaft 180 is converted into oscillation of the rocker shaft 190. To this end, the intermediate link 200 moves in a plane 204 that is perpendicular to the crank axis A3 and the rocker axis A4. The intermediate link 200 is arranged for coordinated movement along this plane 204, which is parallel to the mid-plane.

The ratio of sizes of the crank shaft 180, the rocker shaft 190, and the intermediate link 200 are chosen in order to optimize a transmission angle between the intermediate link 200 and each of the crank shaft 180 and the rocker shaft 190 during operation while maintaining a diameter similar to the handpiece 62. For example, a ratio of a crank stroke 214 and a rocker stroke 216 is approximately 1:2. The crank stroke 214 is the distance between the crank axis A3 and the offset joint 186 of the crank shaft 180, and the rocker stroke 216 is the distance between the rocker axis A4 and the offset joint 196 of the rocker shaft 190.

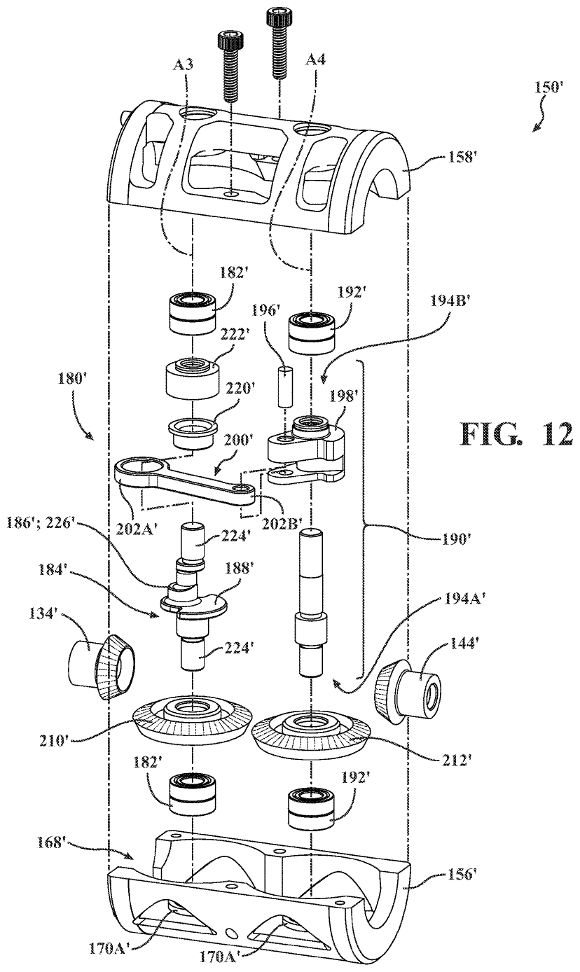

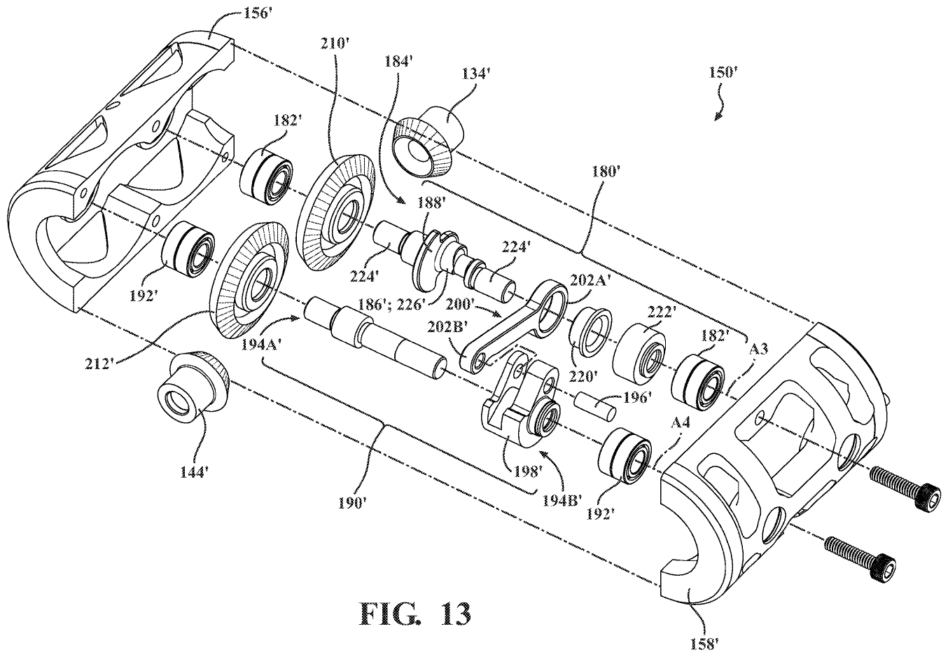

Turning now to FIGS. 12 and 13, an exploded view of a second implementation of the oscillatory motion linkage 150′ is shown. Specifically, the second implementation of the oscillatory motion linkage 150′ includes a second implementation of a crank shaft 180′. As will be appreciated from the subsequent description below, the second crank shaft 180′ is similar to the crank shaft 180 described above in connection with FIGS. 1-11. As such, the components and structural features of the second implementation of the oscillatory motion linkage 150′ that are the same as, or that otherwise correspond to, the first implementation of the oscillatory motion linkage 150 are provided with the same reference numerals with the addition of a prime symbol (e.g., 180 and 180′). While the specific differences between these implementations will be described in detail, for the purposes of clarity, consistency, and brevity, only certain structural features and components common between these implementations will be discussed and depicted in the drawings of the second implementation of the oscillatory motion linkage 150′. Here, unless otherwise indicated, the above description of the first implementation of the oscillatory motion linkage 150 may be incorporated by reference with respect to the second implementation of the oscillatory motion linkage 150′ without limitation.

As with above, the oscillatory motion linkage 150′ comprises the crank shaft 180′, the rocker shaft 190′, and the intermediate link 200′. The crank shaft 180′ is supported for rotation about the crank axis A3 and rotationally coupled to the input shaft via the input gear 134′. Similarly, the rocker shaft 190′ is supported for rotation about the rocker axis A4 and rotationally coupled to the output shaft via the output gear 144′. In the exemplary implementation illustrated herein, the crank axis A3 is parallel to the rocker axis A4. As mentioned above, the crank shaft 180′ is supported in the interior cavity 168′ by the first housing body 156′ and the second housing body 158′ on crank bearings 182′, which are arranged in one of the bearing seats 170′ in each of the first housing body 156′ and the second housing body 158′. Similarly, the rocker shaft 190′ is supported in the interior cavity 168′ by the first housing body 156′ and the second housing body 158′ on rocker bearings 192′, which are arranged in the other one of the bearing seats 170′ in each of the first housing body 156′ and the second housing body 158′.

Here, the second implementation of the crank shaft 180′ may be further defined as a crank shaft assembly 180′. The crank shaft assembly 180′ may comprise a crank portion 184′, a bearing 220′, and a retainer 222′, which are assembled to form the crank shaft assembly 180′. The crank portion 184′ has two journal portions 224′ that define opposing ends of the crank portion 184′, a counterweight portion 188′, and an eccentric portion 226′. The journal portions 224′ are each disposed in one of the crank bearings 182′ for rotationally supporting the crank shaft assembly 180′. The eccentric portion 226′ is axially spaced from the crank axis A3 and cooperates with the bearing 220′ to form the offset joint 186′. The bearing 220′ is assembled to the eccentric portion 226′ to support the intermediate link 200′ for coordinated movement around the crank axis A3. As is discussed above, during operation the offset joint 186′ of the crank shaft assembly 180′ moves in a circular path around the crank axis A3. The counterweight portion 188′ of the crank shaft assembly 180′ is arranged adjacent to and radially opposed from the eccentric portion 226′. The retainer 222′ is coupled to the crank portion 184′ adjacent to the bearing 220′ and opposite the counterweight portion 188′. Said differently, the bearing 220′ is arranged on the eccentric portion 226′ between the counterweight portion 188′ and the retainer 222′. The retainer 222′ facilitates alignment and retention of the bearing 220′ in the link eye 202A′ of the intermediate link 200′, as well as alignment and retention of the bearing 220′ on the eccentric portion 226′ of the crank portion 184′.

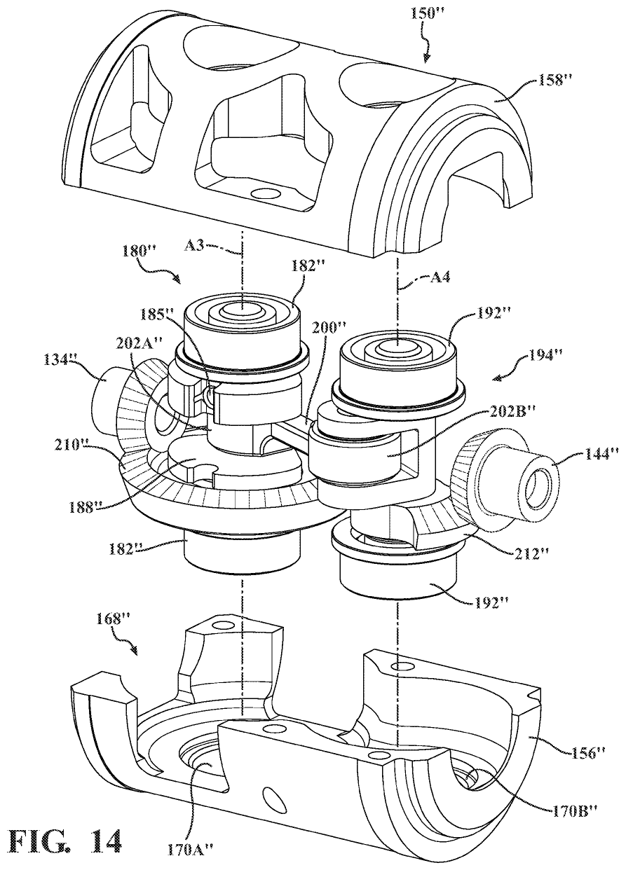

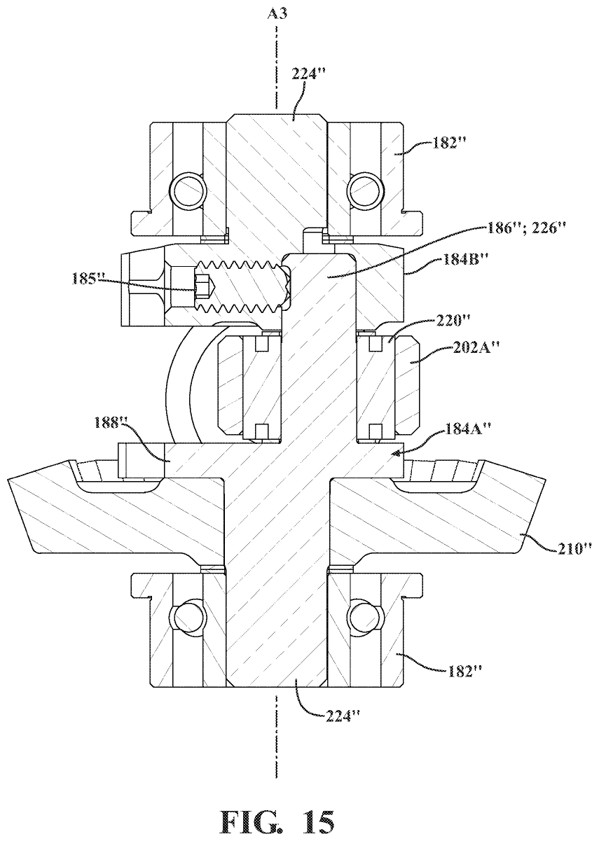

Turning now to FIGS. 14 and 15, a third implementation of the oscillatory motion linkage 150″ is shown. Specifically, the third implementation of the oscillatory motion linkage 150″ includes a third implementation of a crank shaft 180″. As will be appreciated from the subsequent description below, the third crank shaft 180″ is similar to the crank shafts 180 described above in connection with FIGS. 1-11. As such, the components and structural features of the third implementation of the oscillatory motion linkage 150″ that are the same as, or that otherwise correspond to, the first implementation of the oscillatory motion linkage 150 are provided with the same reference numerals with the addition of a second prime symbol (e.g., 180 and 180″). While the specific differences between these implementations will be described in detail, for the purposes of clarity, consistency, and brevity, only certain structural features and components common between these implementations will be discussed and depicted in the drawings of the third implementation of the oscillatory motion linkage 150″. Here, unless otherwise indicated, the above description of the first implementation of the oscillatory motion linkage 150 may be incorporated by reference with respect to the third implementation of the oscillatory motion linkage 150″ without limitation.

As with above, the oscillatory motion linkage 150″ comprises the crank shaft 180″, the rocker shaft 190″, and the intermediate link 200″. The crank shaft 180″ is supported for rotation about the crank axis A3 and rotationally coupled to the input shaft via the input gear 134″. Similarly, the rocker shaft 190″ is supported for rotation about the rocker axis A4 and rotationally coupled to the output shaft via the output gear 144″. In the exemplary implementation illustrated herein, the crank axis A3 is parallel to the rocker axis A4. As mentioned above, the crank shaft 180″ is supported in the interior cavity 168″ by the first housing body 156″ and the second housing body 158″ on crank bearings 182″, which are arranged in one of the bearing seats 170″ in each of the first housing body 156″ and the second housing body 158″. Similarly, the rocker shaft 190″ is supported in the interior cavity 168″ by the first housing body 156″ and the second housing body 158″ on rocker bearings 192″, which are arranged in the other one of the bearing seats 170″ in each of the first housing body 156″ and the second housing body 158″.

Here, the third implementation of the crank shaft 180″ may be further defined as a crank shaft assembly 180″. The crank shaft assembly 180″ may include a first crank portion 184A″, a second crank portion 184B″, and a bearing 220″, which are assembled to form the crank shaft assembly 180″. The first and second crank portions 184A″, 184B″ may be coupled together by a fastener 185″ such as a set screw or a grub screw. In other implementations, the first and second crank portions 184A″, 184B″ may be secured together via interference fit. Using multiple portions is advantageous to allow the eccentric portion to be disposed farther from the axis A3. The first crank portion 184A″ may be assembled to the second crank portion 184B″ to form the offset joint 186″ of the crank shaft 180″. The crank portion 184″ has two journal portions 224″ that define opposing ends of the crank portion 184″, a counterweight portion 188″, and an eccentric portion 226″. The journal portions 224″ are each disposed in one of the crank bearings 182″ for rotationally supporting the crank shaft assembly 180″. The eccentric portion 226″ is axially spaced from the crank axis A3 and cooperates with the bearing 220″ to form the offset joint 186″. The bearing 220″ is assembled to the eccentric portion 226″ to support the intermediate link 200″ for coordinated movement around the crank axis A3. As is discussed above, during operation the offset joint 186″ of the crank shaft assembly 180″ moves in a circular path around the crank axis A3. The counterweight portion 188″ of the crank shaft assembly 180″ is arranged adjacent to and radially opposed from the eccentric portion 226″. It is also contemplated that the rocker shaft 190″ may include a first and a second portion coupled together by a fastener in a similar manner as described above for the crank shaft 180″.

Here, the third implementation of the crank gear 210″ is coupled to the crank shaft 180″ and engaged with the input gear 134″ to transfer rotational movement therebetween. Similarly, a rocker gear 212″ is coupled to the rocker shaft 190″ and engaged with the output gear 144″ to transfer rotational movement therebetween. As with the input gear 134″ and the output gear 144″ described above, the crank gear 210″ and the rocker gear 212″ may be bevel gears. Indeed, in the exemplary implementation illustrated herein, the crank gear 210″ and the rocker gear 212″ are bevel gears configured to transfer mechanical movement (rotation or oscillation, respectively) with the corresponding input gear 134″ and output gear 144″. The crank gear 210″ is configured to continuously rotate about the crank axis A3 while the rocker gear 212″ is configured to oscillate such that the rocker gear 212″ does not make a full rotation about the rocker axis. In some implementations, the rocker gear 212″ may comprise a partial bevel gear as shown in FIG. 14. When the rocker gear 212″ comprises a partial bevel gear, the rocker gear 212″ can accommodate a larger crank gear 210″ and reduce weight.

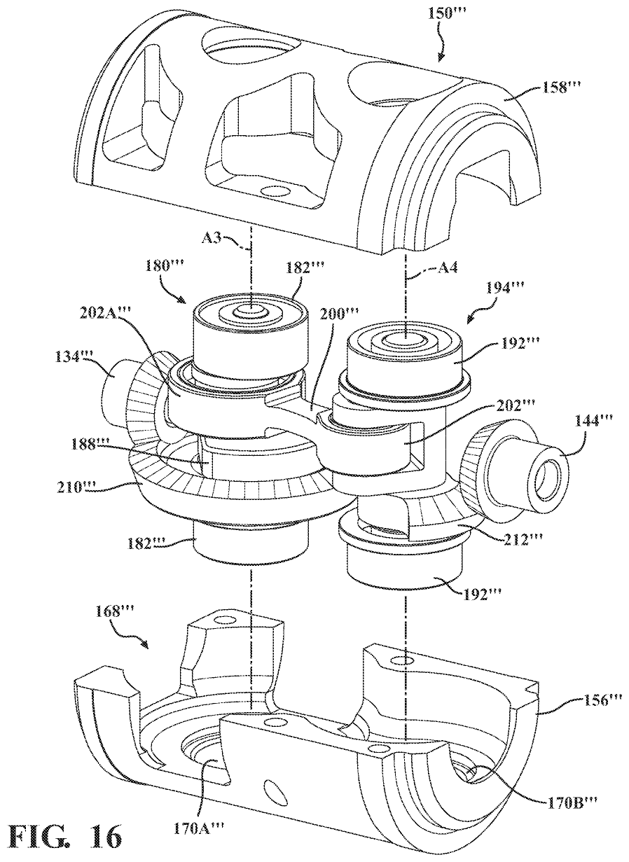

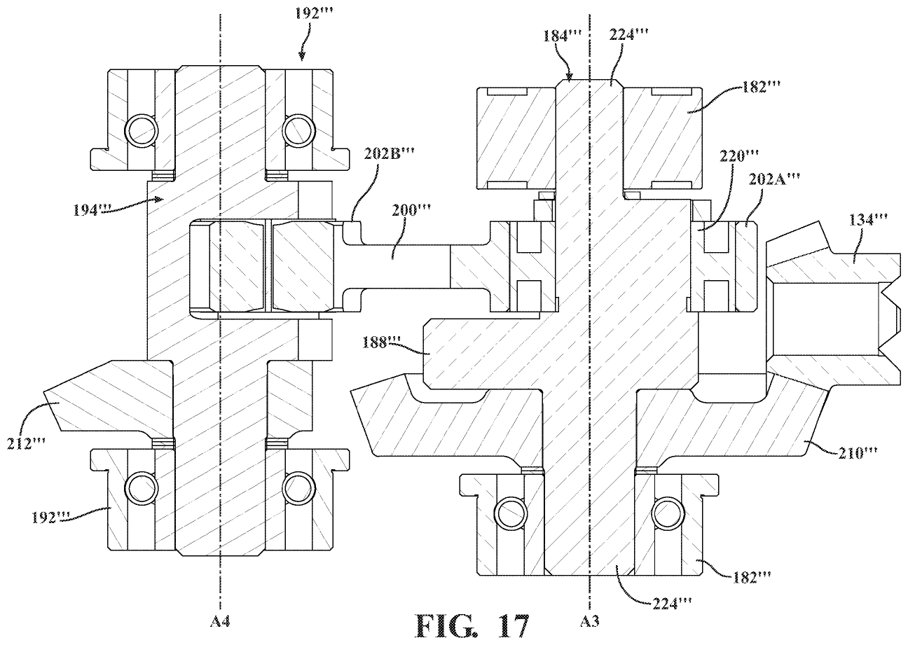

Turning now to FIGS. 16 and 17, a fourth implementation of the oscillatory motion linkage 150′″ is shown. Specifically, the fourth implementation of the oscillatory motion linkage 150″′ includes a fourth implementation of a crank shaft 180″. As will be appreciated from the subsequent description below, the fourth crank shaft 180″′ is similar to the crank shaft 180 described above in connection with FIGS. 1-11. As such, the components and structural features of the fourth implementation of the oscillatory motion linkage 150′″ that are the same as, or that otherwise correspond to, the first implementation of the oscillatory motion linkage 150 are provided with the same reference numerals with the addition of a third prime symbol (e.g., 180 and 180′″). While the specific differences between these implementations will be described in detail, for the purposes of clarity, consistency, and brevity, only certain structural features and components common between these implementations will be discussed and depicted in the drawings of the third implementation of the oscillatory motion linkage 150′″. Here, unless otherwise indicated, the above description of the first implementation of the oscillatory motion linkage 150 may be incorporated by reference with respect to the fourth implementation of the oscillatory motion linkage 150′″ without limitation.

As with above, the oscillatory motion linkage 150′″ comprises the crank shaft 180′″, the rocker shaft 190″′, and the intermediate link 200″′. The crank shaft 180′″ is supported for rotation about the crank axis A3 and rotationally coupled to the input shaft via the input gear 134″′. Similarly, the rocker shaft 190′″ is supported for rotation about the rocker axis A4 and rotationally coupled to the output shaft via the output gear 144″′. In the exemplary implementation illustrated herein, the crank axis A3 is parallel to the rocker axis A4. As mentioned above, the crank shaft 180″′ is supported in the interior cavity 168″′ by the first housing body 156″′ and the second housing body 158″′ on crank bearings 182″′, which are arranged in one of the bearing seats 170″′ in each of the first housing body 156″′ and the second housing body 158″′. Similarly, the rocker shaft 190′″ is supported in the interior cavity 168″′ by the first housing body 156″′ and the second housing body 158″′ on rocker bearings 192″′, which are arranged in the other one of the bearing seats 170″′ in each of the first housing body 156″′ and the second housing body 158″′.

Here, the fourth implementation of the crank shaft 180′″ may be further defined as a crank shaft assembly 180″. The crank shaft assembly 180′″ may comprise a crank portion 184″′ and a bearing 220′″, which are assembled to form the crank shaft assembly 180′″ . The crank portion 184″ has two journal portions 224′″ that define opposing ends of the crank portion 184″′, a counterweight portion 188′″, and an eccentric portion 226′″. The journal portions 224′″ are each disposed in one of the crank bearings 182″′ for rotationally supporting the crank shaft assembly 180″′. The eccentric portion 226″′ is axially spaced from the crank axis A3 and cooperates with the bearing 220′″ to form the offset joint 186″′. In some implementations the crank portion 184′″ may comprise a monolithic construction such that the journal portions 224″′, the eccentric portion 226″′, and the counterweight portion 188″′ comprise a single component. It is contemplated that the rocker shaft 190′″ may also comprise a monolithic construction. The monolithic construction may provide greater rigidity in the shafts 180″′, 190′″ and may mitigate alignment concerns when using multiple components that result from tolerance stack-up. The bearing 220″′ is assembled to the eccentric portion 226′″ to support the intermediate link 200′″ for coordinated movement around the crank axis A3. As is discussed above, during operation the offset joint 186′″ of the crank shaft assembly 180′″ moves in a circular path around the crank axis A3. The counterweight portion 188″′ of the crank shaft assembly 180′″ is arranged adjacent to and radially opposed from the eccentric portion 226″′.

Here, the fourth implementation of the crank gear 210′″ is coupled to the crank shaft 180″′ and engaged with the input gear 134′″ to transfer rotational movement therebetween. Similarly, a rocker gear 212′″ is coupled to the rocker shaft 190′″ and engaged with the output gear 144′″ to transfer rotational movement therebetween. As with the input gear 134′″ and the output gear 144″′ described above, the crank gear 210′″ and the rocker gear 212′″ may be bevel gears. Indeed, in the exemplary implementation illustrated herein the crank gear 210″′ and the rocker gear 212′″ are bevel gears configured to transfer mechanical movement (rotation or oscillation, respectively) with the corresponding input gear 134″′ and output gear 144″′.

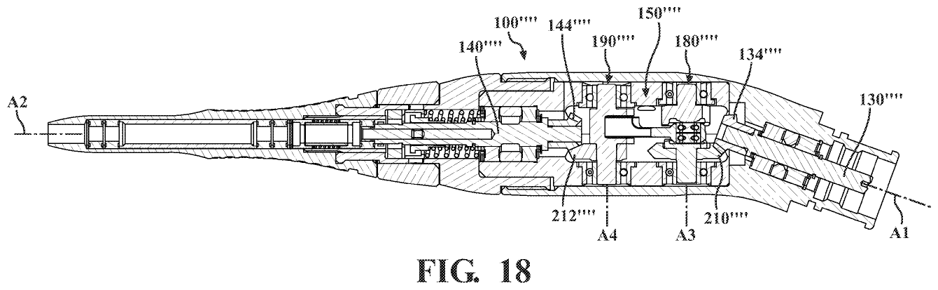

Turning now to FIG. 18, a fifth implementation of the oscillatory motion linkage 150″″ and attachment 100″″ are shown. Specifically, the fifth implementation of the oscillatory motion linkage 150″″ includes a fifth implementation of a crank shaft 180″″. As will be appreciated from the subsequent description below, the fourth crank shaft 180″″ is similar to the crank shaft 180 described above in connection with FIGS. 1-11. As such, the components and structural features of the fifth implementation of the oscillatory motion linkage 150″″ and attachment 100″″ that are the same as, or that otherwise correspond to, the first implementation of the oscillatory motion linkage 150 are provided with the same reference numerals with the addition of a fourth prime symbol (e.g., 180 and 180″″). While the specific differences between these implementations will be described in detail, for the purposes of clarity, consistency, and brevity, only certain structural features and components common between these implementations will be discussed and depicted in the drawings of the third implementation of the oscillatory motion linkage 150″″. Here, unless otherwise indicated, the above description of the first implementation of the oscillatory motion linkage 150 and attachment 100 may be incorporated by reference with respect to the fifth implementation of the oscillatory motion linkage 150″″ and attachment 100″″ without limitation.

As with above, the oscillatory motion linkage 150″″ comprises the crank shaft 180″, the rocker shaft 190″″, and the intermediate link. The crank shaft 180″″ is supported for rotation about the crank axis A3 and rotationally coupled to the input shaft 130″″ via the input gear 134″″. Similarly, the rocker shaft 190″″ is supported for rotation about the rocker axis A4 and rotationally coupled to the output shaft 140″″ via the output gear 144″″. In the exemplary implementation illustrated herein, the crank axis A3 is parallel to the rocker axis A4.

Here, the fifth implementation of the crank gear 210″″ is coupled to the crank shaft 180″″ and engaged with the input gear 134″″ to transfer rotational movement therebetween. Similarly, a rocker gear 212″″ is coupled to the rocker shaft 190″″ and engaged with the output gear 144″″ to transfer rotational movement therebetween. As with the input gear 134″″ and the output gear 144″″ described above, the crank gear 210″″ and the rocker gear 212″″ may be bevel gears. Indeed, in the exemplary implementation illustrated herein the crank gear 210″″ and the rocker gear 212″″ are bevel gears configured to transfer mechanical movement (rotation or oscillation, respectively) with the corresponding input gear 134″″ and output gear 144″″.

Here, the fifth implementation of the attachment 100″″ is angled such that the input shaft 130′″ and the output shaft 140″″ do not rotate about the same axis. Furthermore, the bevel gears are illustrated having a pitch angle suitable to support a power transfer angle of 45 degrees. It is contemplated that other pitch angles may be utilized to achieve a power transfer angle other than 45 degrees. Said differently, the input shaft 130″″ is configured to rotate about the instrument axis Al when coupled to the motor housing. The output shaft 140″″ is configured to rotate about the drive axis A2. The instrument axis Al and the drive axis A2 are not coaxial, but they do intersect.

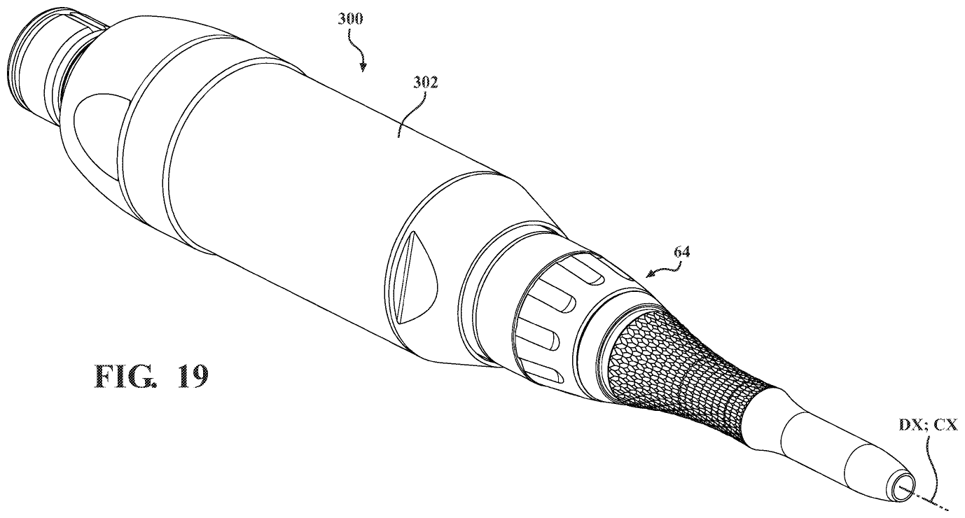

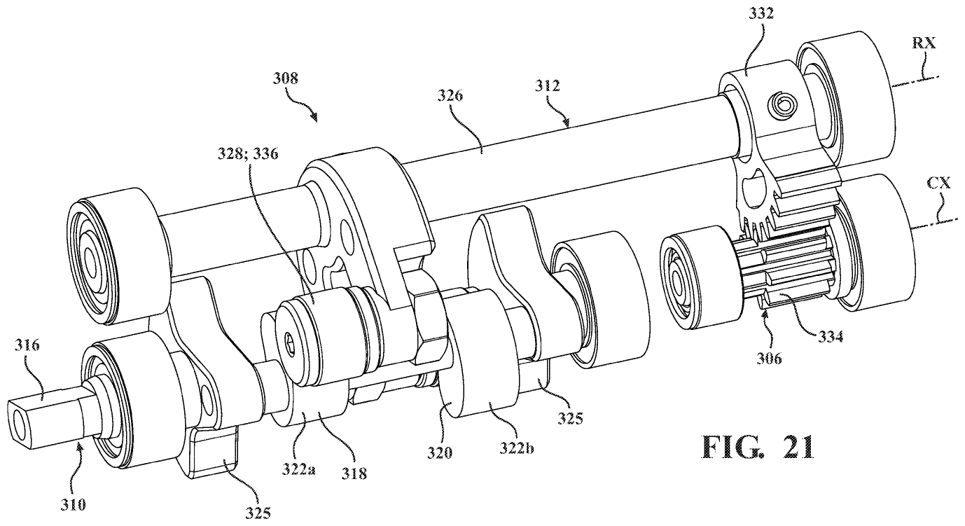

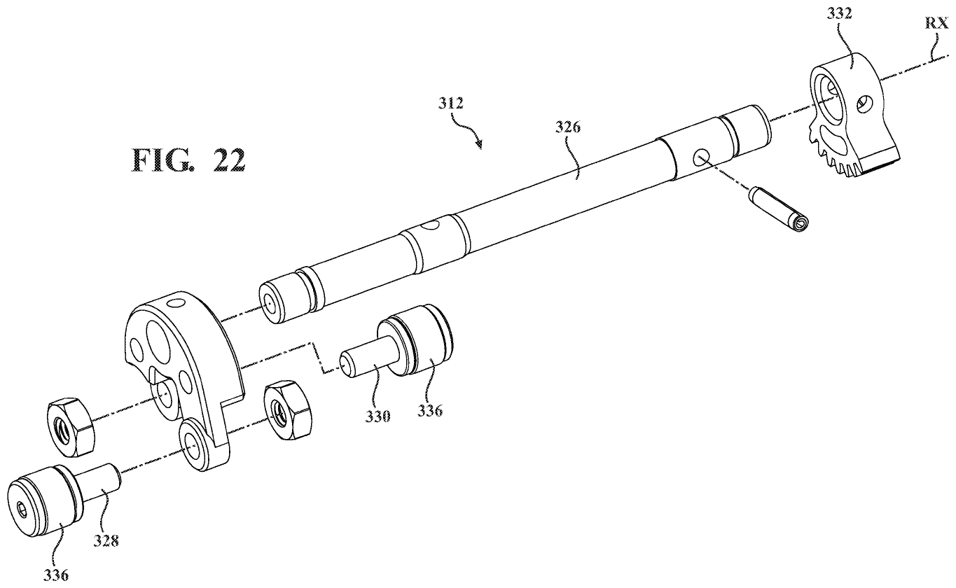

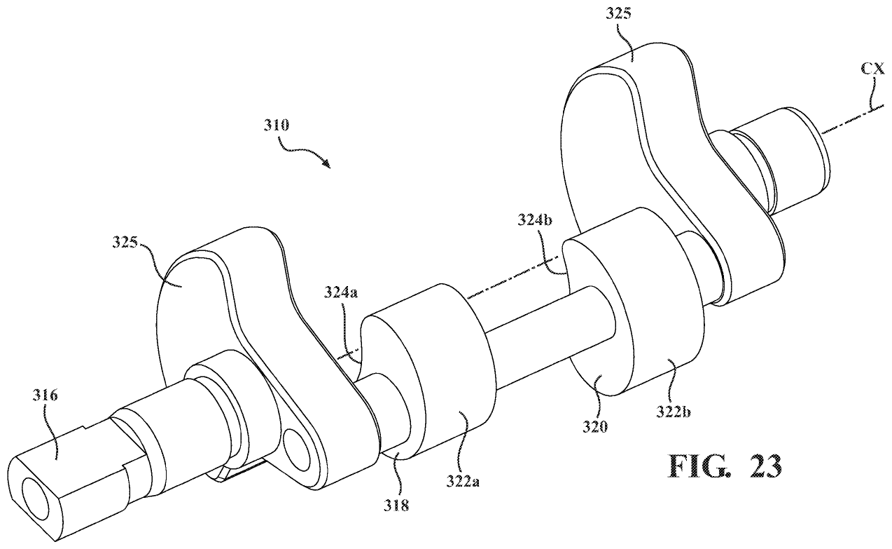

Another implementation of a motion converting attachment 300 is illustrated in FIGS. 19-26C. The attachment may be coupled to the handpiece 62 in a similar manner as the earlier attachment 100. The attachment 300 is also configured to support a nose tube assembly and cutting accessory such as the earlier described nose tube assembly 64 and cutting accessory 56. As with the above-described attachments 100, 100″″ and oscillatory motion linkages 150, 150″, 150′″, 150″″, the attachment 300 transfers rotary mechanical energy received from the handpiece 62 and converts it to oscillatory mechanical energy to be applied to the cutting accessory 56.

The attachment 300 may comprise a housing 302 extending along a drive axis DX between a proximal end and a distal end and define an interior cavity. The attachment 300 may further comprise an input shaft 304 and an output shaft 306 each supported for rotation about the drive axis DX and at least partially disposed in the interior cavity. The input shaft 304 is configured to be coupled to and receive rotational energy from the motor of the handpiece 62. The output shaft 306 is configured to be coupled to the cutting accessory 56 or a drive shaft of the nose tube assembly 64 that in turn couples to the cutting accessory 56 to oscillate the cutting accessory 56. In some instances, the input and output shafts 304, 306 may be supported to rotate about separate axes that are not coaxial. In such instances, the axes about which the input and output shafts 304, 306 rotate may intersect.

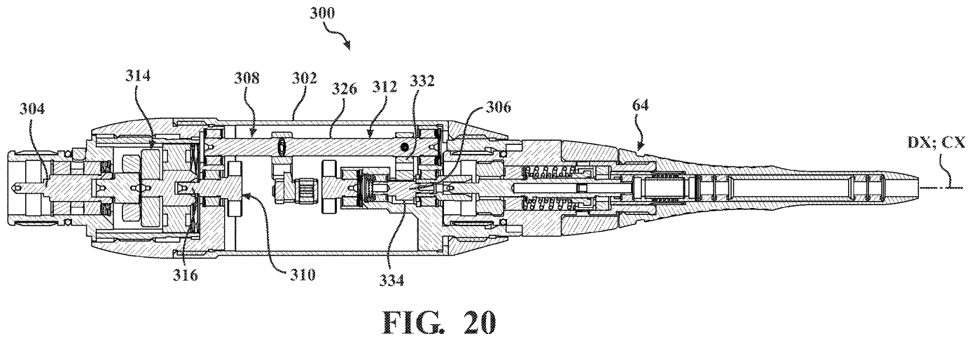

The attachment 300 may further comprise an oscillatory conversion mechanism 308. The oscillatory conversion mechanism 308 comprises a cam shaft 310 and a rocker shaft 312. The cam shaft 310 is supported for rotation about a cam axis CX and rotationally coupled to the input shaft 304 for continuous rotation about the cam axis CX. In the configuration illustrated in FIG. 20, a gear assembly 314 is interposed between the input shaft 304 and the cam shaft 310. The gear assembly 314 may comprise a planetary gear assembly to establish speed reduction between the motor and the output shaft 306 such that the motor makes more than one rotation to result in a single oscillation of the output shaft 306. The gear assembly 314 permits the attachment 300 to be used with high-speed motors that may rotate at speeds greater than 75,000 RPM. In other configurations, the cam shaft 310 is coupled directly to the input shaft 304. In still other configurations, the cam shaft 310 may comprise the input shaft 304 such that the cam shaft 310 connects directly to the handpiece 62. In the configuration illustrated in FIGS. 19-26C, the cam axis CX is coaxial with the drive axis DX to reduce the overall size of the attachment 300. In other configurations, the cam axis CX and the drive axis DX may not be coaxial. The rocker shaft 312 is supported for rotation about a rocker axis RX and rotationally coupled to the output shaft 306 to oscillate the output shaft 306. The rocker axis RX and the cam axis CX are arranged to be parallel to each other.

The cam shaft 310 may include an input 316 to receive torque from the input shaft 304. The cam shaft 310 also includes a clockwise cam lobe 318 and a counter-clockwise cam lobe 320 axially spaced from the clockwise cam lobe 318. In the configuration illustrated in FIGS. 19-26C, the clockwise cam lobe 318 is disposed proximally to the counter-clockwise cam lobe 320. It is contemplated that the cam lobes 318, 320 may be arranged in an opposite orientation. The cam lobes 318, 320 are configured to revolve about the cam axis CX in the same direction relative to each other as the cam shaft 310 is continuously rotated about the cam axis CX. The profile of the cam lobes 318, 320 may be identical to each other with arrangements of the cam lobes 318, 320 being opposite. Each of the cam lobes 318, 320 has a convex surface 322a, 322b to engage the rocker shaft 312 and a concave surface 324a, 324b to allow the rocker shaft 312 to be in clearance with the rocker shaft 312. The cam shaft 310 may include balancing masses 325 to balance the cam shaft 310 during rotation about the cam axis CX. In some configurations, the cam lobes 318, 320 are spaced from the cam axis CX such that no portion of the cam lobes 318, 320 intersect the cam axis CX during rotation of the cam shaft 310 about the cam axis CX. Spacing the cam lobes 318, 320 farther from the cam axis CX can assist with establishing large oscillation angles available at the output shaft 306.

The rocker shaft 312 includes a rocker member 326 that is configured to rotate about the rocker axis RX. The rocker shaft 312 further includes a first cam follower 328 extending from the rocker member 326 for abutting the clockwise cam lobe 318 and a second cam follower 330 axially spaced from the first cam follower 328 and extending from the rocker member 326 for abutting the counter-clockwise cam lobe 320. The rocker shaft 312 further includes a spur gear 332 distal to the first and second cam followers 328, 330 to engage a pinion portion 334 of the output shaft 306. To reduce the overall size of the attachment 300, a portion of the rocker shaft 312, such as the cam followers 328, 330, may pass through the cam axis CX during rotation of the cam shaft 310. Each of the cam followers 328, 330 may comprise a needle bearing 336 to engage the cam lobes 318, 320. The needle bearings 336 reduce friction, and thus heat generation, between the rocker shaft 312 and the cam shaft 310 by supporting rolling friction between the cam shaft 310 and the rocker shaft 312 instead of sliding friction.

During rotation, the cam lobes 318, 320 alternatingly abut the first and second cam followers 328, 330, respectively, to oscillate the rocker shaft 312 and the spur gear 332 about the rocker axis RX. More specifically, the clockwise cam lobe 318 does not abut the first cam follower 328 while the counter-clockwise cam lobe 320 abuts the second cam follower 330 and the counter-clockwise cam lobe 320 does not abut the second cam follower 330 while the clockwise cam lobe 318 abuts the first cam follower 328. Said differently, the cam lobes 318, 320 do not engage the respective cam followers 328, 330 at the same time. During one rotation of the cam shaft 310, the output shaft 306 may make one full oscillation.

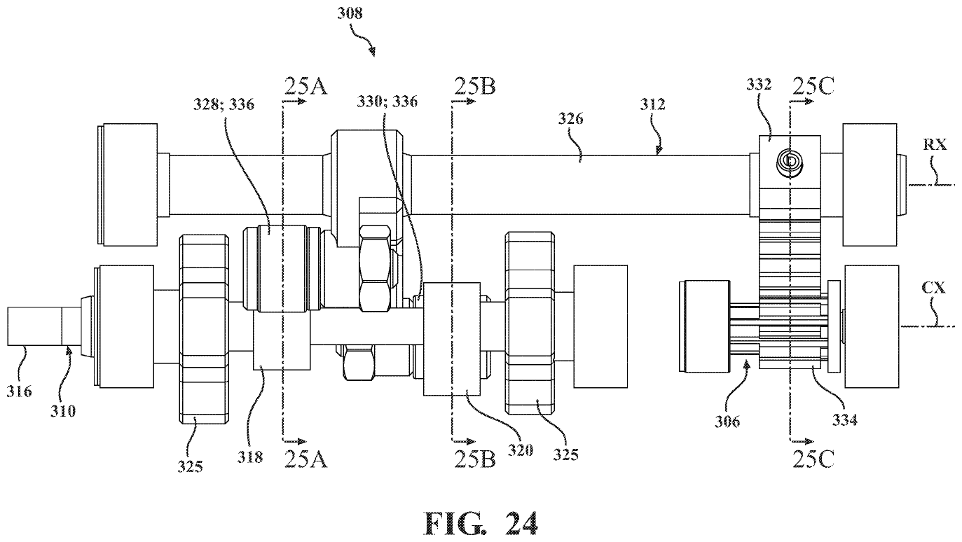

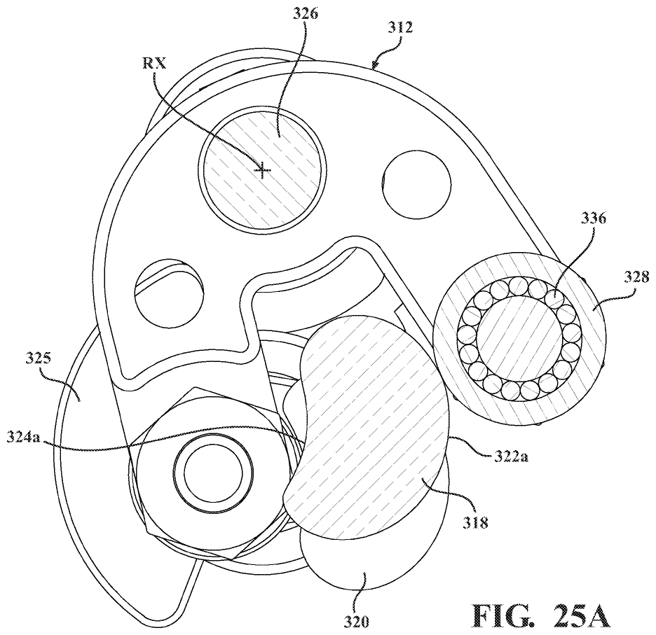

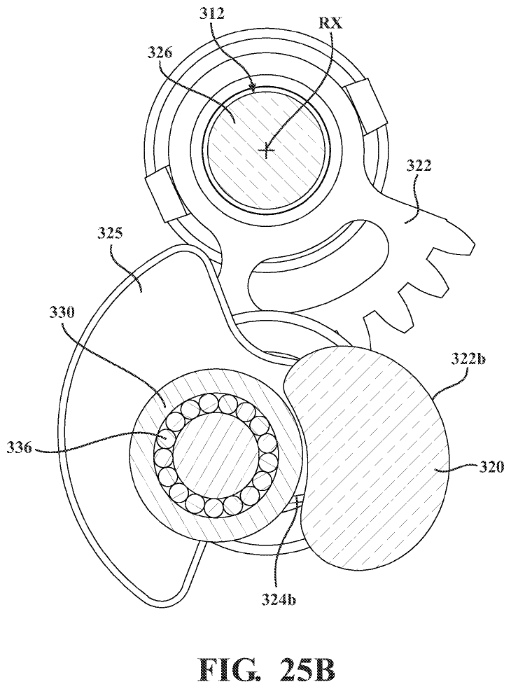

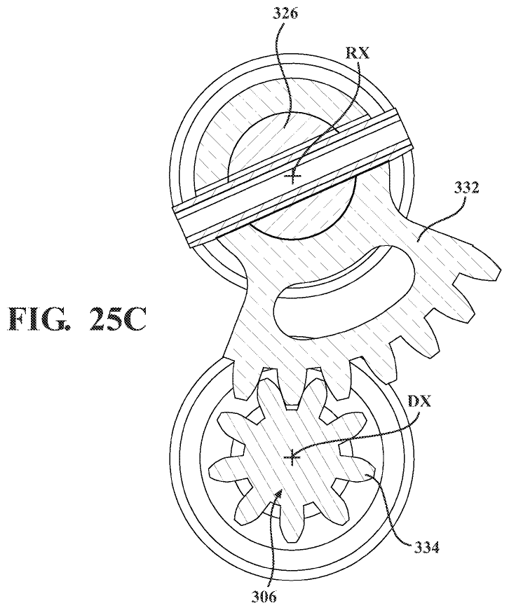

In an exemplary configuration and with reference to FIGS. 24-26C, positions of the cam lobes 318, 320, the cam followers 328, 330, and the spur gear 332 during rotation of the cam shaft 310 are shown. As the cam shaft 310 rotates in one direction about the cam axis CX, the convex surface 322a of the clockwise cam lobe 318 engages the first cam follower 328 to rotate the rocker shaft 312 in the counter-clockwise direction as shown in FIG. 25A, which causes opposite clockwise rotation of the output shaft 306 owing to the meshing engagement between the spur gear 332 and the output shaft 306. As shown in FIG. 25B, while the convex surface 322a of the clockwise cam lobe 318 engages the first cam follower 328, the concave surface 324b of the counter-clockwise cam lobe 320 faces and is in clearance with the second cam follower 330 such that a gap is established therebetween.

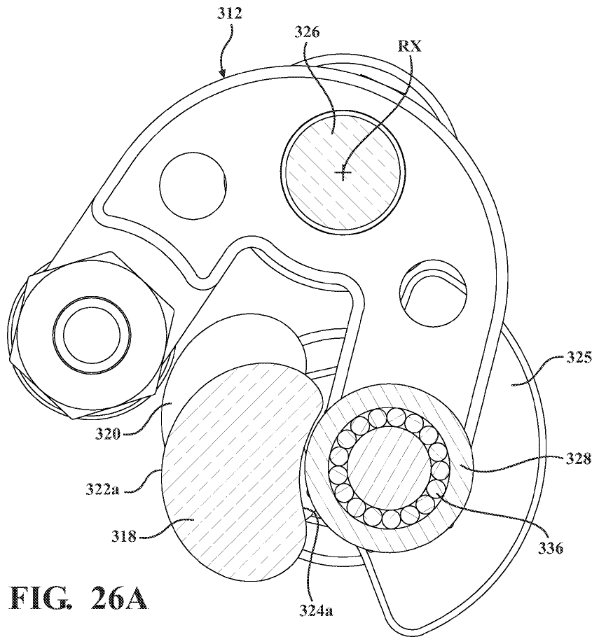

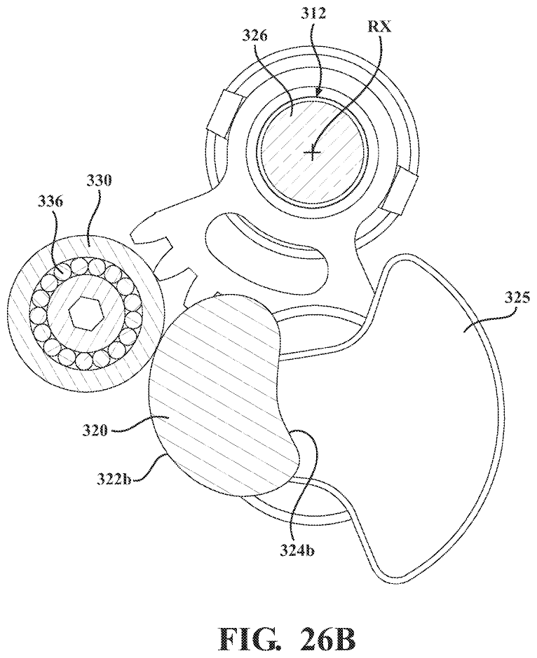

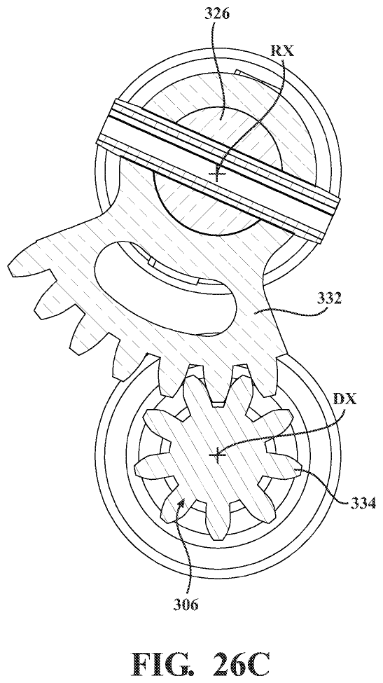

With continued rotation of the cam shaft 310 from the positions shown in FIGS. 25A-25C in the same direction about the cam axis CX, the cam shaft 310 rotates until the clockwise cam lobe 318 is in clearance with the first cam follower 328. After the clockwise cam lobe 318 is in clearance with the first cam follower 328, the convex surface 322b of the counter-clockwise cam lobe 320 engages the second cam follower 330 to rotate the rocker shaft 312 in the clockwise direction as shown in FIG. 26B, which causes opposite counter-clockwise rotation of the output shaft 306 owing to the meshing engagement between the spur gear 332 and the output shaft 306. As shown in FIG. 26A, while the convex surface 322b of the counter-clockwise cam lobe 320 engages the second cam follower 330, the concave surface 324b of the clockwise cam lobe 318 faces and is in clearance with the first cam follower 328 such that a gap is established therebetween.

While clearance between the cam lobes 318, 320 and the cam followers 328, 330 during transition from engagement of one cam lobe 318, 320 to the next may be necessary to prevent binding and assist in assembly, it is appreciated that the clearance may be mitigated to reduce potential chatter or vibration between components.

Several instances have been discussed in the foregoing description. However, the aspects discussed herein are not intended to be exhaustive or limit the disclosure to any particular form. Various modifications to these aspects will be readily apparent to those skilled in the art, and the generic principles defined herein may be applied to other aspects without departing from the scope of the disclosure. The terminology that has been used is intended to be in the nature of words of description rather than of limitation. Many modifications and variations are possible in light of the above teachings and the disclosure may be practiced otherwise than as specifically described.

### CLAUSES

I. An attachment for a rotary surgical tool comprising: a housing extending along a drive axis between a proximal end and a distal end and defining an interior cavity; an input shaft and an output shaft each supported for rotation about the drive axis and at least partially disposed in the interior cavity; a crank shaft supported for rotation about a crank axis and rotationally coupled to the input shaft, wherein the crank axis is perpendicular to the drive axis; a rocker shaft supported for rotation about a rocker axis and rotationally coupled to the output shaft; and an intermediate link coupled between the crank shaft and the rocker shaft.
II. The attachment of clause I, further comprising: an input gear coupled to a distal end of the input shaft; and a crank gear coupled to the crank shaft and engaged with the input gear to transfer rotational movement therebetween.
III. The attachment of clause II, further comprising: an output gear coupled to a proximal end of the output shaft; and a rocker gear coupled to the rocker shaft and engaged with the output gear to transfer rotational movement therebetween.
IV. The attachment of clause III, wherein a ratio between the input gear and the crank gear is approximately 2:1, and wherein a ratio between the rocker gear and the output gear is approximately 2:1.
V. The attachment of clause III, wherein the input gear, the output gear, the crank gear, and the rocker gear are bevel gears.
VI. The attachment of clause I, wherein the housing comprises a first housing body and a second housing body, and wherein the first housing body and the second housing body are coupled at a mid-plane parallel to the drive axis.
VII. The attachment of clause VI, further comprising: an input gear coupled to a distal end of the input shaft; and a crank gear coupled to the crank shaft and engaged with the input gear to transfer rotational movement therebetween, wherein the crank gear is arranged on the crank shaft across the mid-plane from the intermediate link.
VIII. The attachment of clause I, wherein the crank shaft comprises an offset joint axially spaced from the crank axis, and wherein a first end of the intermediate link is coupled to the offset joint.
IX. The attachment of clause VIII, wherein the crank shaft comprises a counterweight radially opposed from the offset joint.
X. The attachment of clause VIII, wherein the rocker shaft comprises an offset joint axially spaced from the rocker axis, and wherein a second end of the intermediate link is coupled to the offset joint of the rocker shaft.
XI. The attachment of clause X, wherein a distance between the crank axis and the offset joint of the crank shaft is further defined as a crank stroke and a distance between the rocker axis and the offset joint of the rocker shaft is further defined as a rocker stroke, and wherein a ratio between the crank stroke and the rocker stroke is approximately 1:2.
XII. The attachment of clause X, wherein the rocker shaft comprises a counterweight radially opposed from the offset joint of the rocker shaft.
XIII. An attachment for a rotary surgical tool comprising: a housing extending between a proximal end and a distal end and defining an interior cavity; an input shaft supported for rotation in the housing and having an input bevel gear arranged at a distal end of the input shaft; an output shaft supported for rotation in the housing and having an output bevel gear arranged at a proximal end of the output shaft; an oscillatory motion linkage operably coupled between the input shaft and the output shaft, the oscillatory motion linkage comprising: a crank shaft supported for rotation about a crank axis; a rocker shaft supported for rotation about a rocker axis; an intermediate link coupled between the crank shaft and the rocker shaft and configured to convert rotational movement of the crank shaft to oscillatory movement of the rocker shaft; a crank bevel gear coupled to the crank shaft and engaged with the input bevel gear; and a rocker bevel gear coupled to the rocker shaft and engaged with the output bevel gear.
XIV. The attachment of clause XIII, wherein the housing comprises a first housing body and a second housing body, and wherein the first housing body and the second housing body are coupled at a mid-plane perpendicular to the crank axis.
XV. The attachment of clause XIV, wherein the input shaft rotates about a drive axis, and wherein the drive axis is parallel to the mid-plane.
XVI. The attachment of clause XIV, wherein the crank bevel gear is arranged on the crank shaft across the mid-plane from the intermediate link.
XVII. The attachment of clause XIII, wherein the crank shaft comprises an offset joint axially spaced from the crank axis, and wherein a first end of the intermediate link is coupled to the offset joint.
XVIII. The attachment of clause XVII, wherein the crank shaft comprises a counterweight radially opposed from the offset joint.
XIX. The attachment of clause XVII, wherein rocker shaft comprises an offset joint axially spaced from the rocker axis, and wherein a second end of the intermediate link is coupled to the offset joint of the rocker shaft.
XX. The attachment of clause XIX, wherein a distance between the crank axis and the offset joint of the crank shaft is further defined as a crank stroke and a distance between the rocker axis and the offset joint of the rocker shaft is further defined as a rocker stroke, and wherein a ratio between the crank stroke and the rocker stroke is approximately 1:2.
XXI. The attachment of clause XIX, wherein the rocker shaft comprises a counterweight radially opposed from the offset joint of the rocker shaft.
XXII. An attachment for a rotary surgical tool comprising: a housing assembly extending along a drive axis between a proximal end and a distal end, the housing assembly comprising: a first housing body; a second housing body coupled to the first housing body at a mid-plane, wherein the mid-plane is parallel to the drive axis; and an interior cavity defined by the first housing body and the second housing body; an input shaft and an output shaft each supported for rotation about the drive axis and at least partially disposed in the interior cavity; a crank shaft rotationally coupled to the input shaft; a rocker shaft rotationally coupled to the output shaft; and an intermediate link coupled between the crank shaft and the rocker shaft and arranged for coordinated movement along a plane parallel to the mid-plane.
XXIII. The attachment of clause XXII, further comprising: an input gear coupled to a distal end of the input shaft; and a crank gear coupled to the crank shaft and engaged with the input gear to transfer rotational movement therebetween.
XXIV. The attachment of clause XXIII, wherein the crank shaft comprises an offset joint axially spaced from an axis of rotation of the crank shaft, and wherein a first end of the intermediate link is coupled to the offset joint.
XXV. The attachment of clause XXIV, wherein the crank gear is arranged on the crank shaft across the mid-plane from the offset joint.
XXVI. The attachment of clause XXIV, wherein the crank shaft comprises a counterweight radially opposed from the offset joint.
XXVII. The attachment of clause XXIV, further comprising: an output gear coupled to a proximal end of the output shaft; and a rocker gear coupled to the rocker shaft and engaged with the output gear to transfer rotational movement therebetween.
XXVIII. The attachment of clause XXVII, wherein rocker shaft comprises an offset joint axially spaced from an axis of rotation of the rocker shaft, and wherein a second end of the intermediate link is coupled to the offset joint of the rocker shaft.
XXIX. The attachment of clause XXVIII, wherein the rocker shaft comprises a counterweight radially opposed from the offset joint of the rocker shaft.
XXX. The attachment of clause XXVIII, wherein the rocker gear is arranged on the rocker shaft across the mid-plane from the offset joint of the rocker shaft.
XXXI. The attachment of clause XXVIII, wherein a distance between the axis of rotation of the crank shaft and the offset joint of the crank shaft is further defined as a crank stroke and a distance between the axis of rotation of the rocker shaft and the offset joint of the rocker shaft is further defined as a rocker stroke, and wherein a ratio between the crank stroke and the rocker stroke is approximately 1:2.
XXXII. The attachment of clause XXVII, wherein a ratio between the input gear and the crank gear is approximately 2:1, and wherein a ratio between the rocker gear and the output gear is approximately 2:1.
XXXIII. The attachment of clause XXVII, wherein the input gear, the output gear, the crank gear, and the rocker gear are bevel gears.
XXXIV. An attachment for a rotary surgical tool comprising: a housing extending between a proximal end and a distal end and defining an interior cavity; an input shaft supported for rotation in the housing; an output shaft supported in the housing coaxial to the input shaft; and an oscillatory motion linkage operably coupled between the input shaft and the output shaft and disposed in the interior cavity, wherein the oscillatory motion linkage is configured to convert rotational motion of the input shaft to oscillating motion of the output shaft.
XXXV. A surgical bur system comprising: a rotary surgical handpiece comprising a motor, an attachment interface, and a motor shaft arranged in the attachment interface for supplying rotational motion from the motor; an oscillatory motion attachment removably couplable with the rotary surgical handpiece, the oscillatory motion attachment comprising: a housing extending between a proximal end and a distal end and configured to engage the attachment interface; an input shaft supported for rotation in the housing and arranged to engage the motor shaft when the housing is engaged with the attachment interface; an output shaft supported in the housing coaxial to the input shaft; an oscillatory motion linkage operably coupled between the input shaft and the output shaft and disposed in the interior cavity, wherein the oscillatory motion linkage is configured to convert rotational motion of the input shaft to oscillating motion of the output shaft; and a nose tube assembly removably coupled to the oscillatory motion attachment opposite the rotary surgical handpiece, the nose tube assembly comprising a drive shaft arranged to engage the output shaft.

1. An attachment for a rotary surgical tool comprising:
a housing extending along a drive axis between a proximal end and a distal end and defining an interior cavity;
an input shaft and an output shaft each supported for rotation about the drive axis and at least partially disposed in the interior cavity;
a crank shaft supported for rotation about a crank axis and rotationally coupled to the input shaft, wherein the crank axis is perpendicular to the drive axis;
a rocker shaft supported for rotation about a rocker axis and rotationally coupled to the output shaft; and
an intermediate link coupled between the crank shaft and the rocker shaft.

2. The attachment of claim 1, further comprising:
an input gear coupled to a distal end of the input shaft; and
a crank gear coupled to the crank shaft and engaged with the input gear to transfer rotational movement therebetween.

3. The attachment of claim 2, further comprising:
an output gear coupled to a proximal end of the output shaft; and
a rocker gear coupled to the rocker shaft and engaged with the output gear to transfer rotational movement therebetween.

4. The attachment of claim 3, wherein a ratio between the input gear and the crank gear is approximately 2:1, and wherein a ratio between the rocker gear and the output gear is approximately 2:1.

5. The attachment of claim 3, wherein the input gear, the output gear, the crank gear, and the rocker gear are bevel gears.

6. The attachment of claim 1, wherein the housing comprises a first housing body and a second housing body, and wherein the first housing body and the second housing body are coupled at a mid-plane parallel to the drive axis.

7. The attachment of claim 6, further comprising:
an input gear coupled to a distal end of the input shaft; and
a crank gear coupled to the crank shaft and engaged with the input gear to transfer rotational movement therebetween, wherein the crank gear is arranged on the crank shaft across the mid-plane from the intermediate link.

8. The attachment of claim 1, wherein the crank shaft comprises an offset joint axially spaced from the crank axis, and wherein a first end of the intermediate link is coupled to the offset joint.

9. The attachment of claim 8, wherein the crank shaft comprises a counterweight radially opposed from the offset joint.

10. The attachment of claim 8, wherein the rocker shaft comprises an offset joint axially spaced from the rocker axis, and wherein a second end of the intermediate link is coupled to the offset joint of the rocker shaft.

11. The attachment of claim 10, wherein a distance between the crank axis and the offset joint of the crank shaft is further defined as a crank stroke and a distance between the rocker axis and the offset joint of the rocker shaft is further defined as a rocker stroke, and wherein a ratio between the crank stroke and the rocker stroke is approximately 1:2.

12. The attachment of claim 10, wherein the rocker shaft comprises a counterweight radially opposed from the offset joint of the rocker shaft.

13. An attachment for a rotary surgical tool comprising:
a housing extending between a proximal end and a distal end and defining an interior cavity;
an input shaft supported for rotation in the housing and having an input bevel gear arranged at a distal end of the input shaft;
an output shaft supported for rotation in the housing and having an output bevel gear arranged at a proximal end of the output shaft;
an oscillatory motion linkage operably coupled between the input shaft and the output shaft, the oscillatory motion linkage comprising:
a crank shaft supported for rotation about a crank axis;
a rocker shaft supported for rotation about a rocker axis;
an intermediate link coupled between the crank shaft and the rocker shaft and configured to convert rotational movement of the crank shaft to oscillatory movement of the rocker shaft;
a crank bevel gear coupled to the crank shaft and engaged with the input bevel gear; and
a rocker bevel gear coupled to the rocker shaft and engaged with the output bevel gear.

14. The attachment of claim 13, wherein the housing comprises a first housing body and a second housing body, and wherein the first housing body and the second housing body are coupled at a mid-plane perpendicular to the crank axis.

15. The attachment of claim 14, wherein the input shaft rotates about a drive axis, and wherein the drive axis is parallel to the mid-plane.

16. The attachment of claim 14, wherein the crank bevel gear is arranged on the crank shaft across the mid-plane from the intermediate link.

17. The attachment of claim 13, wherein the crank shaft comprises an offset joint axially spaced from the crank axis, and wherein a first end of the intermediate link is coupled to the offset joint.

18. The attachment of claim 17, wherein the crank shaft comprises a counterweight radially opposed from the offset joint.

19. The attachment of claim 17, wherein rocker shaft comprises an offset joint axially spaced from the rocker axis, and wherein a second end of the intermediate link is coupled to the offset joint of the rocker shaft.

20. The attachment of claim 19, wherein a distance between the crank axis and the offset joint of the crank shaft is further defined as a crank stroke and a distance between the rocker axis and the offset joint of the rocker shaft is further defined as a rocker stroke, and wherein a ratio between the crank stroke and the rocker stroke is approximately 1:2.

21. The attachment of claim 19, wherein the rocker shaft comprises a counterweight radially opposed from the offset joint of the rocker shaft.

22-55. (canceled)
